(19) United States
(12) Patent Application Publication
(10) Pub. No.: US 2019/0209501 A1
(43) Pub. Date: Jul. 11, 2019

(54) COMPOSITIONS AND METHODS OF USE OF BETA-HYDROXY-BETA-METHYLBUTYRATE (HMB) ASSOSIATED WITH INTERMITTENT FASTING

(71) Applicants: Texas Tech University Office of Research Commercialization, Lubbock, TX (US); Metabolic Technologies, Inc., Ames, IA (US)

(72) Inventors: Grant Tinsley, M., Lubbock, TX (US); John Rathmacher, Story City, IA (US); Lisa Pitchford, Ames, IA (US)

(21) Appl. No.: 16/240,211

(22) Filed: Jan. 4, 2019

Related U.S. Application Data
(60) Provisional application No. 62/613,952, filed on Jan. 5, 2018.

(51) Int. Cl. A61K 31/19; A61P 3/04; A61P 21/00; A23L 33/10

(52) U.S. Cl. CPC A61K 31/19; A61P 3/04; A61K 9/0053; A23L 33/10; A61P 21/00

(57) ABSTRACT

The present invention provides a composition comprising HMB and methods of using HMB to mitigate loss of lean body mass, increase fat free mass, improve muscular performance, increase body fat loss and decrease body fat percentage in individuals undergoing intermittent fasting.

This application claims priority to United States Provisional Patent Application No. 62/613,952 filed Jan. 5, 2018 and herein incorporates the provisional application by reference.

### BACKGROUND OF THE INVENTION

### 1. Field

The present invention relates to a composition comprising β-hydroxy-β-methylbutyrate (HMB) and methods of using the composition in association with intermittent fasting (IF) to mitigate loss of lean body mass, increase fat free mass, improve muscular performance, increase body fat loss and decrease body fat percentage.

### 2. Background

The increasing prevalence of obesity is a major health crisis. It is projected that by 2030, around 50% of the US adult population will be obese, with major consequences for increases in type 2 diabetes (T2D), cardio-vascular disease (CVD), hypertension, and many cancers. There is a lack of effective long term therapeutic approaches, consequently alternative methods are continuously being investigated for the management of obesity, with limited success. One well-studied approach of intermittent fasting (IF), called alternate-day fasting (ADF), prescribes a schedule of alternating between days of unrestricted food consumption and modified fasting days, during which a single meal of approximately 500 kcal is consumed. The ability of ADF to reduce food consumption, improve body composition and beneficially modify a variety of cardiovascular and metabolic health markers has been repeatedly demonstrated.

Intermittent fasting (IF) is a broad term encompassing eating patterns with regularly-occurring periods of food abstention longer than a typical overnight fast (1). In contrast to traditional methods of continuous energy restriction. IF programs utilize intermittent energy restriction by interspersing periods of less-restricted or unrestricted feeding with periods of severely limited energy intake. Several forms of IF have been described, including time-restricted feeding (TRF) (restricting food intake to specific time periods of the day, typically between 8 to 12 hours each day), alternate-day fasting (ADF) (alternating between no calories for one day and eating without restriction the next), alternate-day modified fasting (alternating between few calories one day and eating without restriction the next) and periodic fasting (fasting 1 or 2 days per week and consuming food ad libitum on 5 to 6 days per week) (2). The vast majority of existing research in humans has focused on weight loss and health effects induced by IF in overweight and obese adults. Cumulatively, this research has demonstrated that IF programs are viable alternatives to traditional continuous energy restriction for weight loss and health improvement (3-5).

Dietary recommendations for fat loss typically involve daily calorie restriction, meaning that a normal eating schedule and frequency is followed but smaller portions and/or fewer calories are consumed at each meal. Intermittent fasting, or employing repeated short-term fasts, works to reduce food consumption, modify body composition and improve overall health. These short term fasts are longer than a typical overnight fast, but are typically no longer than 24 hours in duration.

Intentional reductions in energy intake are frequently implemented by the general population and athletes alike, typically for the goal of fat loss. One important consideration associated with such hypocaloric dietary conditions is the ability to maintain, or slow the loss of, lean body mass. Not only is lean mass critical for functional ability and athletic performance, but reductions in lean mass my drive overeating and promote the regain of fat mass following weight loss. Additionally, maintaining lean mass could lead to superior maintenance of energy expenditure due to its large contribution to resting metabolic rate. Therefore, optimal fat loss programs should promote maximal retention of lean body mass.

In addition to traditional concerns of retaining lean body mass during hypocaloric conditions, IF programs implement fasting periods that necessitate periods of 12 to 24 hours without protein consumption. During this time, it is expected that muscle protein breakdown exceeds muscle protein synthetic activity, thus resulting in a negative protein balance in skeletal muscle. Skeletal muscle tissue may be broken down in short-term fasting in order to provide amino acid substrate for hepatic gluconeogenesis. Despite these concerns, it has previously been demonstrated that resistance training can prevent the loss of lean body mass during IF programs utilizing 16 to 20 hour fasting periods. However, periods of detraining in athletes and known difficulties meeting physical activity requirements in the general population necessitate the exploration of non-exercise strategies to ameliorate a potential loss of skeletal muscle tissue during fat loss programs, including IF.

While an increasing body of research has reported the physiological effects of IF, a very limited number of controlled trials have taken place in active or exercising individuals (6-8). Two previous investigations reported the effects of TRF in adult males performing resistance training (RT) (7, 8). While Tinsley et al. (7) observed an apparent attenuation in lean mass accretion during 8 weeks of TRF, this result was confounded by the TRF group self-selecting a protein intake lower than the control diet (1.0 vs. 1.4 g/kg/d) and suboptimal for active individuals (9, 10). Nonetheless, comparable improvements in muscular performance were observed in both groups. Moro et al. (8) prescribed higher protein intake (1.9 g/kg/d) in TRF and control diets and found that, while both groups maintained lean mass and demonstrated similar muscular performance, TRF produced significant reductions in fat mass (FM) and differential effects on physiological markers.

The prevalence of IF eating patterns in active individuals and the paucity of existing research in this population indicate the need for further research. Additionally, no previous trials have examined the effects of IF plus RT in females, despite some reports identifying potential sex differences in responses to IF in humans (11, 12). Furthermore, since IF programs necessitate prolonged periods without amino acid-induced stimulation of muscle protein synthesis and suppression of muscle protein breakdown (13), it has been questioned whether modification of fasting periods to allow ingestion of amino acids or their metabolites may be beneficial for lean mass maintenance or accretion during IF (14). However, no previous trials have examined this empirically. Therefore, the studies described below were designed to compare the physiological and performance effects of TRF, with or without supplementation of the leucine metabolite beta-hydroxy beta-methylbutyrate (HMB) during fasting periods, to a control diet requiring breakfast consumption during progressive RT in active females.

One important concern associated with all weight loss programs, including intermittent fasting, including ADF, is the potential loss of lean body mass (LBM). While a number of recent ADF trials have demonstrated fat mass loss and beneficial health improvements, losses of LBM have also been reported. Due to the large contribution of LBM to resting metabolic rate and functional abilities, it is critical to develop weight loss strategies that minimize the LBM loss while maximizing fat mass reductions. It has recently demonstrated that resistance training (RT) can reduce the loss of LBM, often seen during intermittent fasting and it has also demonstrated that the combination of ADF and aerobic exercise produce greater weight and fat loss than either individual treatment. However, none of these strategies were sufficient to completely reverse the associated losses of LBM.

HMB

Alpha-ketoisocaproate (KIC) is the first major and active metabolite of leucine. A minor product of KIC metabolism is β-hydroxy-β-methylbutyrate (HMB). HMB has been found to be useful within the context of a variety of applications. Specifically, in U.S. Pat. No. 5,360,613 (Nissen), HMB is described as useful for reducing blood levels of total cholesterol and low-density lipoprotein cholesterol. In U.S. Pat. No. 5,348,979 (Nissen et al.), HMB is described as useful for promoting nitrogen retention in humans. U.S. Pat. No. 5,028,440 (Nissen) discusses the usefulness of HMB to increase lean tissue development in animals. Also, in U.S. Pat. No. 4,992,470 (Nissen), HMB is described as effective in enhancing the immune response of mammals. U.S. Pat. No. 6,031,000 (Nissen et al.) describes use of HMB and at least one amino acid to treat disease-associated wasting.

The use of HMB to suppress proteolysis originates from the observations that leucine has protein-sparing characteristics. The essential amino acid leucine can either be used for protein synthesis or transaminated to the α-ketoacid (α-ketoisocaproate. KIC). In one pathway. KIC can be oxidized to HMB and this accounts for approximately 5% of leucine oxidation. HMB is superior to leucine in enhancing muscle mass and strength. The optimal effects of HMB can be achieved at 3.0 grams per day when given as calcium salt of HMB, or 0.038 g/kg of body weight per day, while those of leucine require over 30.0 grams per day.

Once produced or ingested, HMB appears to have two fates. The first fate is simple excretion in urine. After HMB is fed, urine concentrations increase, resulting in an approximate 20-50% loss of HMB to urine. Another fate relates to the activation of HMB to HMB-CoA. Once converted to HMB-CoA, further metabolism may occur, either dehydration of HMB-CoA to MC-CoA, or a direct conversion of HMB-CoA to HMG-CoA, which provides substrates for intracellular cholesterol synthesis. Several studies have shown that HMB is incorporated into the cholesterol synthetic pathway and could be a source for new cell membranes that are used for the regeneration of damaged cell membranes. Human studies have shown that muscle damage following intense exercise, measured by elevated plasma CPK (creatine phosphokinase), is reduced with HMB supplementation within the first 48 hrs. The protective effect of HMB lasts up to three weeks with continued daily use. Numerous studies have shown an effective dose of HMB to be 3.0 grams per day as CaHMB (calcium HMB) (˜38 mg·kg body weight⁻¹·day⁻¹). HMB has been tested for safety, showing no side effects in healthy young or old adults. HMB in combination with L-arginine and L-glutamine has also been shown to be safe when supplemented to AIDS and cancer patients.

Recently, HMB free acid, a new delivery form of HMB, has been developed. This new delivery form has been shown to be absorbed quicker and have greater tissue clearance than CaHMB. The new delivery form is described in U.S. Patent Publication Serial No. 20120053240 which is herein incorporated by reference in its entirety.

HMB has been demonstrated to enhance recovery and attenuate muscle damage from high intensity exercise. HMB attenuates the depression of protein synthesis with TNF-alpha and decreases protein degradation associated with TNF.

HMB is effective in reducing muscle protein breakdown and promoting muscle protein synthesis, translating into increased LBM and improved muscle function in both young and older adult populations, during health and disease. Further, HMB has been demonstrated in U.S. patent application Ser. No. 15/170,329 that consuming HMB results in reductions in fat mass and increased fat loss.

It has been surprisingly and unexpectedly discovered that administration of HMB mitigates the loss of LBM during intermittent fasting induced weight loss to a greater extent than resistance training alone, thereby enhancing maintenance of metabolic rate. It has also been discovered that administration of HMB with an intermittent fasting program results in greater losses of fat as compared to participation in an intermittent fasting program alone. Further, the fat loss associated with administration of HMB and an intermittent fasting program is greater than the fat loss associated with administration of HMB alone.

It has been discovered that fat free mass gain is greater with intermittent fasting and HMB administration over intermittent fasting or control diet. In addition, resting metabolic rate increases with intermittent fasting and HMB while it decreases with control diet and intermittent fasting groups.

It has also been discovered that cortisol is decreased with HMB supplementation during acute fasting (a single 24-hour fast). HMB supplementation modifies the cortisol awakening response by producing a more rapid reduction in cortisol concentrations. HMB supplementation also alters the testosterone:cortisol ratio in males.

### SUMMARY OF THE INVENTION

One object of the present invention is to provide a composition for in conjunction with intermittent fasting to mitigate the loss of lean body mass.

Another object of the present invention is to provide a composition to improve muscular performance in individuals undergoing fasting.

A further object of the present invention is to provide methods of administering a composition in association with intermittent fasting to increase body fat loss and/or decrease body fat percentage.

An additional object of the present invention is to provide methods of administering a composition in association with intermittent fasting to increase fat-free mass.

A further object of the present invention is to provide methods of administering a composition in association with intermittent fasting to increase the resting metabolic rate.

These and other objects of the present invention will become apparent to those skilled in the art upon reference to the following specification, drawings, and claims.

The present invention intends to overcome the difficulties encountered heretofore. To that end, a composition comprising HMB is provided. The composition is administered to a subject in need thereof. The composition is consumed by a subject in need thereof. All methods comprise administering to the animal HMB. The subjects included in this invention include humans and non-human mammals.

### BRIEF DESCRIPTION OF THE DRAWINGS

FIG. 1 is a table showing body composition changes.

FIG. 2 is a table showing muscular performance changes.

### DETAILED DESCRIPTION OF THE INVENTION

It has been surprising and unexpectedly discovered that HMB administered during a period of reduced food consumption, such as intermittent fasting (IF) mitigates the loss of lean body mass that results from reduced food consumption. Intermittent fasting employs repeated short-term fasts, which are longer than a typical overnight fast but typically shorter than 24 hours in duration in an effort to reduce food consumption. These fasting periods are alternated with unrestricted feeding periods and may be implemented every day, every other day, or even one day per week.

Consumption of HMB can be used in conjunction with any intermittent fasting period, including but not limited to alternate-day fasting (ADF), which prescribes a schedule of alternating between days of unrestricted food consumption and modified fasting days, during which a single meal is consumed or time restricted feeding (TRF). Intermittent fasting has been demonstrated to reduce food consumption, improve body composition and beneficially modify a variety of cardiovascular and metabolic health markers. HMB can also be used in conjunction with acute fasting.

One important concern associated with all weight loss programs, including intermittent fasting, is the associated loss of LBM, commonly observed with significant fat mass loss and accompanied by beneficial health improvements. Due to the large contribution of LBM to resting metabolic rate and functional abilities, it is critical to develop weight loss strategies that minimize LBM loss while maximizing fat mass reductions. It has been demonstrated that RT can reduce LBM often seen during IF. Additionally, it has also demonstrated that the combination of intermittent fasting and aerobic exercise produces greater weight and fat loss than either individual treatment. However, many individuals find it difficult to adhere to an exercise program, and most Americans do not meet the recommended physical activity recommendations. Therefore, while exercise should be encouraged as part of weight loss programs, there is also a great need for additional interventions (either adjuvant to minimal exercise, or completely non-exercise in nature) that can preserve LBM during weight loss programs, such as intermittent fasting. In accordance with this invention, HMB is one such intervention used to preserve LBM during intermittent fasting. HMB supplementation mitigates the loss of LBM during intermittent fasting induced weight loss to a greater extent than resistance training alone, thereby enhancing maintenance of metabolic rate and fat mass reductions. In addition, it was surprisingly and unexpectedly discovered that HMB supplementation in conjunction with an intermittent fasting program resulting it fat loss, and that this fat loss was significantly greater than that seen when using HMB alone.

β-hydroxy-β-methylbutyric acid, or β-hydroxy-isovaleric acid, can be represented in its free acid form as (CH₃)₂(OH)CCH₂COOH. The term “HMB” refers to the compound having the foregoing chemical formula, in both its free acid and salt forms, and derivatives thereof. While any form of HMB can be used within the context of the present invention, preferably HMB is selected from the group comprising a free acid, a salt, an ester, and a lactone. HMB esters include methyl and ethyl esters. HMB lactones include isovalaryl lactone. HMB salts include sodium salt, potassium salt, chromium salt, calcium salt, magnesium salt, alkali metal salts, and earth metal salts.

Methods for producing HMB and its derivatives are well-known in the art. For example, HMB can be synthesized by oxidation of diacetone alcohol. One suitable procedure is described by Coffman et al., J. Am. Chem. Soc. 80: 2882-2887 (1958). As described therein, HMB is synthesized by an alkaline sodium hypochlorite oxidation of diacetone alcohol. The product is recovered in free acid form, which can be converted to a salt. For example, HMB can be prepared as its calcium salt by a procedure similar to that of Coffman et al. (1958) in which the free acid of HMB is neutralized with calcium hydroxide and recovered by crystallization from an aqueous ethanol solution. The calcium salt of HMB is commercially available from Metabolic Technologies, Ames. Iowa.

### Calcium β-Hydroxy-β-Methylbutyrate (HMB) Supplementation

More than 2 decades ago, the calcium salt of HMB was developed as a nutritional supplement for humans. Studies have shown that 38 mg of CaHMB per kg of body weight appears to be an efficacious dosage for an average person.

The molecular mechanisms by which HMB decreases protein breakdown and increases protein synthesis have been reported. Eley et al conducted in vitro studies which have shown that HMB stimulates protein synthesis through mTOR phosphorylation. Other studies have shown HMB decreases proteolysis through attenuation of the induction of the ubiquitin-proteosome proteolytic pathway when muscle protein catabolism is stimulated by proteolysis inducing factor (PIF), lipopolysaccharide (LPS), and angiotensin II. Still other studies have demonstrated that HMB also attenuates the activation of caspases-3 and -8 proteases.

### HMB Free Acid Form

In most instances, the HMB utilized in clinical studies and marketed as an ergogenic aid has been in the calcium salt form. Recent advances have allowed the HMB to be manufactured in a free acid form for use as a nutritional supplement. Recently, a new free acid form of HMB was developed, which was shown to be more rapidly absorbed than CaHMB, resulting in quicker and higher peak serum HMB levels and improved serum clearance to the tissues.

HMB free acid may therefore be a more efficacious method of administering HMB than the calcium salt form, particularly when administered directly preceding intense exercise. One of ordinary skill in the art, however, will recognize that this current invention encompasses HMB in any form.

HMB in any form may be incorporated into the delivery and/or administration form in a fashion so as to result in a typical dosage range of about 0.5 grams HMB to about 30 grams HMB.

Any suitable dose of HMB can be used within the context of the present invention. Methods of calculating proper doses are well known in the art. The dosage amount of HMB can be expressed in terms of corresponding mole amount of Ca-HMB. The dosage range within which HMB may be administered orally or intravenously is within the range from 0.01 to 0.2 grams HMB (Ca-HMB) per kilogram of body weight per 24 hours. For adults, assuming body weights of from about 100 to 200 lbs., the dosage amount orally or intravenously of HMB (Ca-HMB basis) can range from 0.5 to 30 grams per subject per 24 hours.

When the composition is administered orally in an edible form, the composition is preferably in the form of a dietary supplement, foodstuff or pharmaceutical medium, more preferably in the form of a dietary supplement or foodstuff. Any suitable dietary supplement or foodstuff comprising the composition can be utilized within the context of the present invention. One of ordinary skill in the art will understand that the composition, regardless of the form (such as a dietary supplement, foodstuff or a pharmaceutical medium), may include amino acids, proteins, peptides, carbohydrates, fats, sugars, minerals and/or trace elements.

In order to prepare the composition as a dietary supplement or foodstuff, the composition will normally be combined or mixed in such a way that the composition is substantially uniformly distributed in the dietary supplement or foodstuff. Alternatively, the composition can be dissolved in a liquid, such as water.

The composition of the dietary supplement may be a powder, a gel, a liquid or may be tabulated or encapsulated. In addition to HMB, the composition may include other components, including vitamins (such as vitamin D, vitamin B, vitamin C, etc.), amino acids delivered in the free form (such as arginine, glutamine, lysine, etc.) and/or via protein, carbohydrates, fats, etc.

Although any suitable pharmaceutical medium comprising the composition can be utilized within the context of the present invention, preferably, the composition is combined with a suitable pharmaceutical carrier, such as dextrose or sucrose.

Furthermore, the composition of the pharmaceutical medium can be intravenously administered in any suitable manner. For administration via intravenous infusion, the composition is preferably in a water-soluble non-toxic form. Intravenous administration is particularly suitable for hospitalized patients that are undergoing intravenous (IV) therapy. For example, the composition can be dissolved in an IV solution (e.g., a saline or glucose solution) being administered to the patient. Also, the composition can be added to nutritional IV solutions, which may include amino acids, glucose, peptides, proteins and/or lipids. The amounts of the composition to be administered intravenously can be similar to levels used in oral administration. Intravenous infusion may be more controlled and accurate than oral administration.

Methods of calculating the frequency by which the composition is administered are well-known in the art and any suitable frequency of administration can be used within the context of the present invention (e.g., one 6 g dose per day or two 3 g doses per day) and over any suitable time period (e.g., a single dose can be administered over a five minute time period or over a one hour time period, or, alternatively, multiple doses can be administered over an extended time period). The composition can be administered over an extended period of time, such as weeks, months or years.

Any suitable dose of HMB can be used within the context of the present invention. Methods of calculating proper doses are well known in the art.

The term administering or administration includes providing a composition to a mammal, consuming the composition and combinations thereof.

### EXPERIMENTAL EXAMPLES

The following examples will illustrate the invention in further detail. It will be readily understood that the composition of the present invention, as generally described and illustrated in the Examples herein, could be synthesized in a variety of formulations and dosage forms. Thus, the following more detailed description of the presently preferred embodiments of the methods, formulations and compositions of the present invention are not intended to limit the scope of the invention, as claimed, but it is merely representative of the presently preferred embodiments of the invention. For example, it is understood that the invention is not limited to the amounts of the composition administered or the form. Effective amounts of HMB are well known in the art and it is recognized that the composition is effective at all points across the range of 0.5 grams to 30 grams of HMB per day, as exemplified by the experimental examples.

### Experimental Example 1

Design

This study employed a randomized, placebo-controlled, reduced factorial design and was double-blind with respect to supplementation in TRF groups. Active females were randomized to control diet (CD), TRF or TRF plus 3 g/d HMB (TRF_{HMB}). TRF groups consumed all calories in ˜8 h/d. All groups completed 8 weeks of supervised RT and consumed supplemental whey protein. Body composition, muscular performance, dietary intake, physical activity, and physiological variables were assessed. Data were analyzed prior to unblinding using mixed models and both per protocol (PP) and intention-to-treat (ITT) frameworks.

Participants and Methods

### Overview

This study employed a randomized, placebo-controlled, reduced factorial design. The experiment was double-blind with respect to HMB and placebo supplements and single-blind when possible with respect to the assigned dietary program. The following primary outcome measures were specified a priori: FM, fat-free mass (FFM), body fat percentage (BF %), muscle thickness of the elbow flexor muscles (MT_{EF}) and muscle thickness of the knee extensor muscles (MT_{KE}). Secondary outcome measures specified a priori included metrics of muscular performance, resting metabolism, blood markers, blood pressure, arterial stiffness, physical activity level and questionnaire responses.

### Participants

Healthy female participants between the ages of 18 and 30 were recruited via posters, email announcements and word of mouth. Participants were required to have prior RT experience, defined as reporting ≥1 year of RT at a frequency of 2 to 4 sessions per week and with weekly training of major upper and lower body muscle groups. Additionally, participants were screened for BF % using multi-frequency bioelectrical impedance analysis (MFBIA; mBCA 514/515, Seca, Hamburg. Germany). The original target BF % range for participants was 15 to 29%; however, due to data from our lab indicating overestimations of body fat via MFBIA as compared to a 4-component model in resistance-trained females (15), individuals with up to 33% body fat at screening were considered eligible. Individuals were excluded if they did not meet the aforementioned criteria or were pregnant, trying to become pregnant, currently breastfeeding, cigarette smokers, allergic to dairy protein or had a pacemaker or other electrical implant. Eligible participants were stratified based on body fat percentage at screening (15 to 21% vs. >21%) and habitual breakfast consumption (≥5 d/week vs. <5 d/week), then randomly assigned to one of the three study groups (control diet plus placebo [CD], TRF plus placebo [TRF] or TRF plus HMB [TRF_{HMB}]) using sequences produced from a random sequence generator (http://www.random.org) and based on a 1:1:1 allocation ratio. Each participant within a given stratum was allocated in a sequential manner to the first available group assignment at the time of baseline testing using the random integer sequence for that stratum. Generation of random sequences and implementation of stratified randomization were performed by the primary investigator (GMT).

### Nutrition and Supplementation Program

Participants in TRF and TRF_{HMB} were instructed to consume all calories between noon and 8 PM each day, and CD participants were instructed to consume breakfast as soon as possible after waking and continue to eat at self-selected intervals throughout the remainder of the day. In addition to the assigned eating schedule, participants were provided with a minimal amount of dietary advice based on the results of their weighed diet records and metabolism testing. Specifically, participants were instructed to consume the provided whey protein supplement (Elite 100% Whey, Dymatize Enterprises, LLC, Dallas, Tex. USA) in order to achieve a protein intake ≥1.4 g/kg/d. This range was chosen based on protein intake recommendations for lean mass accretion or retention in exercising individuals (9). The energy content of supplemental protein was ˜200-250 kcal/d. In all groups, target energy intake was prescribed by multiplying resting energy expenditure (REE) via indirect calorimetry by an activity factor of 1.5, then subtracting 250 kcal. The goal of the small caloric reduction was to promote fat loss while still providing adequate nutritional support for muscular hypertrophy. Prior to commencement of the intervention, as well as during two separate weeks during the intervention, weighed diet records were completed for weekday and weekend days. Each participant was provided with a food scale and instructed how to properly weigh and record food items. The resultant dietary records were manually analyzed by reviewing nutrition facts labels and utilizing the United States Department of Agriculture (USDA) Food Composition Databases (https://ndb.nal.usda.gov/ndb/).

In a double-blind manner, participants in TRF and TRF_{HMB} received placebo (calcium lactate) or calcium HMB supplements, respectively. HMB and placebo capsules were produced by the same manufacturer (Metabolic Technologies, Inc., Ames, Iowa, USA), were identical in appearance and taste, and were matched for calcium (102 mg), phosphorus (26 mg) and potassium (49 mg) content. TRF and TRF_{HMB} participants were instructed to ingest two capsules on three occasions each day: upon waking, mid-morning while still fasting, and prior to bed, for a total dose of 3 g/d. Participants in CD also received the placebo capsules for consumption at breakfast, lunch, and dinner using a unique supplement code to maintain blinding of researchers with respect to the supplements used in TRF and TRF_{HMB}. All researchers were blinded to the supplement assignments of the TRF groups until after data collection and statistical analysis were completed, at which time the study sponsor provided supplement codes for unblinding. Additionally, trainers supervising the RT program were asked not to discuss group assignment with participants in order to maintain blinding. Participants were discouraged from consuming any additional sports supplements beyond those provided by study investigators, with the exception of common multi-vitamin/mineral supplements.

### Resistance Training Program and Physical Activity Monitoring

All groups completed 8 weeks of supervised RT in conjunction with the assigned dietary and supplementation programs. Training took place within the research laboratories under direct supervision. RT sessions were completed on 3 nonconsecutive days each week (i.e. Mondays, Wednesdays and Fridays), and upper- and lower-body sessions were alternated (Table 1).

TABLE 1
Resistance Training Program.

| Upper Body A | | | Lower Body A | | | Upper Body B | | | Lower Body B | | |
|---|---|---|---|---|---|---|---|---|---|---|---|
| | 0-4 | 4-8 | | 0-4 | 4-8 | | 0-4 | 4-8 | | 0-4 | 4-8 |
| Exercise | W | W | Exercise | W | W | Exercise | W | W | Exercise | W | W |
| Bentover | 4 × 8-12, | 5 × 6-8, | BB | 4 × 8-12, | 5 × 6-8, | BB bench | 4 × 8-12, | 5 × 6-8, | BB back | 4 × 8-12, | 5 × 6-8, |
| DB rows | 120 s | 180 s | deadlift | 120 s | 180 s | press | 120 s | 180 s | squat | 120 s | 180 s |

| DB bench | 4 × 8-12, 120 s | Hip sled | 4 × 8-12, | 5 × 6-8, | Bentover | 4 × 8-12, 120 s | Stiff-leg | 4 × 8-12, | 5 × 6-8, |
|---|---|---|---|---|---|---|---|---|---|
| press | | | 120 s | 180 s | DB rows | | deadlift | 120 s | 180 s |
| BB shoulder | 4 × 8-12, 120 s | Lunges | 4 × 8-12, 120 s | | DB shoulder | 4 × 8-12, 120 s | Lunges | 4 × 8-12, 120 s | |
| press | | with DB | | | press | | with DB | | |
| DB flyes | 4 × 8-12, 90 s | Leg curls | 4 × 8-12, 90 s | | DB curls | 4 × 8-12, 90 s | Leg curls | 4 × 8-12, 90 s | |
| Preacher | 4 × 8-12, 90 s | Leg | 4 × 8-12, 90 s | | Skull- | 4 × 8-12, 90 s | Leg | 4 × 8-12, 90 s | |
| curls | | extensions | | | crushers | | extensions | | |
| Triceps | 4 × 8-12, 90 s | | | | Inverted rows | 4 × 8-12, 90 s | | | |
| extension | | | | | (bodyweight) | | | | |

Exercise prescription shown as. sets × repetition range, rest interval.
BB: barbell: DB: dumbbell: s: seconds: W: weeks

Participants were instructed to train to momentary muscular exhaustion for each set, and the load was adjusted as necessary to ensure compliance with the specified repetition range. The weights and repetitions completed for each set of each exercise were recorded to allow for calculation of RT volume. Sessions took place between 12:00 and 18:00. Participants in TRF and TRF_{HMB} who performed RT sessions between 12:00 and 13:00 were asked to shift their feeding window one hour earlier (i.e. 11:00 to 19:00) on training days to ensure that RT did not take place in the fasted state. Following each RT session, participants from each group were provided with 25 g whey protein (Elite 100% Whey, Dymatize Enterprises, LLC, Dallas, Tex. USA).

Participants were asked not to perform any RT outside of the study intervention, as well as to avoid other high-intensity exercise. In order to objectively assess free-living physical activity levels during the course of intervention, each participant was provided with an accelerometer (ActiGraph GT9X Link; Actigraph Inc. Pensacola, Fla. USA) at baseline, during the first half of the intervention and during the second half of the intervention. Participants were instructed to wear the devices during waking hours, whenever they were not bathing or sleeping, for at least 4 days. The accelerometer was set to record accelerations at a sampling rate of 30 Hz. and accelerations were converted into activity counts per 1-min epoch length during post data processing. The activity counts data were screened for determining wear time for each monitoring day where non-wear time was defined as a period with ≥60 min of consecutive zero activity counts (i.e., no movement), with an allowance up to 2 minutes of interruption with activity counts <100 per minute (16). Physical activity energy expenditure (PAEE; kcal/min) was estimated for each minute of wear time using the Freedson's prediction equation (17) for activity counts >1951 counts per minute and the Williams Work-Energy equation for activity counts ≥1951 counts per minute (18). Daily PAEE was averaged across valid days of each participant where a valid day was defined as a day with ≥10 hours of wear time. Lastly, although the estimated non-wear time were assumed to be non-waking hours, average daily PAEE was adjusted by average wear time for each participant using a least-square adjustment method (19) due to the possibility of misclassification influencing daily PAEE.

### Overview of Laboratory Assessments

At baseline and after 4 and 8 weeks of the intervention, participants completed two testing sessions: (1) a morning assessment conducted after an overnight fast for assessment of body composition, metabolism, vascular measures and subjective factors: and (2) an afternoon assessment of muscular performance, conducted in the non-fasted state. For morning assessments, participants reported to the laboratory after abstention from eating, drinking, exercising and utilizing caffeine or nicotine for ≥8 hours. Participants were interviewed to confirm adherence to these pre-assessment restrictions. The actual abstention from exercise was ≥14 hours due to the scheduling of exercise sessions. Participants reported to the laboratory wearing athletic clothing, and all metal and accessories were removed from the body prior to testing. Each participant voided her bladder and provided a urine sample. Urine samples were assessed for urine specific gravity (USG) using a digital refractometer (PA201X-093. Misco, Solon, Ohio. USA). Additionally, a standard urinary HCG test was performed to confirm that each participant was not pregnant. Finally, urinary samples were frozen at −80° C. for assessment of urinary HMB content after study unblinding. After voiding, each participant's body mass (BM) and height were determined via digital scale with stadiometer (Seca 769, Hamburg, Germany). Blood draws were performed at Texas Tech University Student Health Services after an overnight fast, and participants completed at-home saliva collections for assessment of the cortisol awakening response (CAR).

### Body Composition Assessment

Body composition was assessed using a modified 4-component (4C) model (20, 21) produced from dual-energy x-ray absorptiometry (DXA) and bioimpedance spectroscopy (BIS) data. DXA scans were performed on a Lunar Prodigy scanner (General Electric, Boston, Mass., USA) with enCORE software (v. 16.2). The scanner was calibrated using a quality control block each morning prior to use, and positioning of participants was conducted according to manufacturer recommendations. Each participant was able to fit within the scanning dimensions. DXA bone mineral content (BMC) was divided by 0.9582 to yield an estimate of bone mineral (Mo) (22). Additionally, body volume (BV) was estimated from DXA lean soft tissue (LST), fat mass (FM) and BMC using the equation developed by Wilson et al. for General Electric DXA scanners (20):

BV(L)=0.933*LST+1.150*FM+−0.438*BMC+1.504

BIS was utilized to obtain total body water (TBW) estimates. BIS utilizes Cole modeling (23) and mixture theories (24) to predict body fluids rather than regression equations used by other impedance methods (e.g. bioelectrical impedance analysis (25)). The BIS device used in the present study (SFB7, ImpediMed. Carlsbad, Calif., USA) employs 256 measurement frequencies ranging from 4 to 1,000 kHz. Each participant remained supine for ≥5 minutes immediately prior to assessment using the manufacturer-recommended hand-to-foot electrode arrangement. Duplicate assessments were performed, with the values averaged for analysis. Assessments were reviewed for quality assurance through visual inspection of Cole plots.

The 4C equation of Wang et al. was utilized for estimation of whole-body FM (26):

FM(kg)=2.748*BV−0.699*TBW+1.129*Mo−2.051*BM

FFM was calculated as BM−FM, and BF % was calculated as (FM/BM)×100.

In addition to whole-body composition estimates, muscle thickness of the elbow flexors (MT_{EF}) and knee extensors (MT_{KE}) was evaluated via ultrasonography (Logiq e, General Electric. Boston, Mass. USA) at baseline and study completion. Elbow flexor measurements took place at 66% of the distance from the acromion of the scapula to the cubital fossa, and knee extensor measurements took place at 50% of the distance from the anterior superior iliac spine to the superior border of the patella (27, 28). These distances were measured while the participant was standing, and measurement distances at baseline were recorded and used at the final assessment. All assessments took place on the right side of the body. In the supine position, the participant's arm was abducted to ˜80° with the arm supported for elbow flexor measurements. For knee extensor measurements, a foam pad was placed beneath the knee to allow an ˜10° bend at the knee joint. For all assessments, transmission gel was generously applied to the marked measurement location, and minimal pressure was applied by the transducer in order to avoid tissue compression. Three single transverse images were taken at each location, with values averaged for analysis. The gain and depth of the transducer were kept consistent for all measurements at a given site. Ultrasound images were blinded for analysis and analyzed by a single blinded researcher using ImageJ (v. 1.52a; National Institutes of Health, USA). The reliability of the researcher analyzing ultrasound images was determined through blinded analysis of 28 randomly selected ultrasound images on two occasions. This exercise produced minimum differences (MD) of 0.07 cm for MT_{EF} and 0.14 cm for MT_{KE}.

### Muscular Performance Assessment

Assessments of muscular performance took place between 12:00 and 18:00 in the non-fasted state, and participants were instructed to follow their preferred food and fluid intake patterns prior to testing. The assessment began with a 5-minute warm up period using a self-selected pace on a stationary bicycle. This warm up period was followed by assessment of countermovement vertical jumps (CMVJ) performance, testing on a mechanized squat device, and muscular strength and endurance assessment on the bench press and hip sled exercises. At the 4-week assessment, the CMVJ and hip sled assessments were not performed.

For the CMVJ tests, participants completed eight trials while wearing their own footwear. Approximately 30 seconds rest separated each trial. Ground reaction force (GRF) data were obtained during the CMVJ using two force platforms sampled at 1 kHz (OPT464508; Advanced Mechanical Technology, Inc., Watertown, Mass. USA). Participants stood motionless with each foot positioned on a force platform and their hands on their hips before initiating the CMVJ with a countermovement action using a self-selected depth and jumping with maximum effort to achieve the highest vertical displacement possible. No instructions were provided for the landing phase except to land with each foot contacting its respective force platform from take-off and to stop downward motion and return to a motionless standing position. The raw GRF data from the two force platforms were smoothed using a fourth order low pass Butterworth digital filter with a 30 Hz cutoff frequency. The smoothed GRF from the two force platforms was then summed along the vertical axis to obtain the vertical GRF acting at the body center of mass. The start of the CMVJ was defined as the time when bodyweight was reduced by 2.5% (29). Take-off was defined as the time when the summed vertical GRF decreased below a 20 N threshold (30). Jump time was then calculated as the time elapsed between the start of the CMVJ and take-off, expressed in units of seconds. Vertical jump height was calculated using the impulse-momentum relationship and expressed in units of meters.

Isometric and isokinetic squats were performed using a mechanized squat device (Exerbotics eSq, Tulsa, Okla., USA) (31, 32). At the first assessment, each participant's preferred foot positioning was determined using a custom grid overlaid on the foot platform of the squat device. This foot positioning was recorded and utilized for all visits. No weight belts, knee wraps, or other aids were utilized during testing. Prior to testing, the participant's range of motion for isokinetic testing was determined. The range of motion was set to 90° between the thigh and lower leg at the bottom of the repetition and approximately 170° at the top of the repetition, as determined by a goniometer. The isometric testing included maximal effort pushes at 120° and 150° knee angles. Each participant was instructed to push against the device as hard and fast as possible while attempting to complete a squat movement. Two isometric pushes were performed at each knee angle, and each effort lasted approximately 2 to 3 seconds. After the isometric testing, a 3-repetition maximum isokinetic force production test was completed. Prior to testing, participants observed the movement of the machine and received verbal instruction regarding proper performance of the assessment. Each of the repetitions during the maximal isokinetic force production test consisted of a 4-second eccentric phase, followed by an approximately half-second pause at the 90° knee position and a 4-second concentric phase. During testing, the force signal was sampled from the load cell at 1 kHz (MP100; Biopac Systems, Inc, Santa Barbara, Calif., USA), stored on a personal computer, and processed off-line using custom-written software (LabVIEW, Version 11.0; National Instruments, Austin, Tex., USA). The scaled force signal was low-pass filtered, with a 10-Hz cutoff (zero-phase lag, fourth-order Butterworth filter). All subsequent analyses were conducted on the scaled and filtered force signal. For the isometric force production tests, the rate of force development (RFD) over specific time intervals (i.e. 30, 50, 100 and 200 ms) was calculated by manually specifying the onset of force production within the custom LabVIEW program. For each repetition of the maximum isokinetic force production test, isokinetic peak forces (PF) were determined as the highest mean 25-ms epoch for both concentric and eccentric testing (i.e. PF_{CONC} and PF_{ECC}).

Resistance exercise performance for the bench press and hip sled exercises was evaluated via the 1-repetition maximum (1RM) and repetitions to failure with 70% of the 1RM. The 1RM testing protocol was based on the recommendations of the National Strength and Conditioning Association (33). Briefly, after completing warm up sets, participants completed 2 to 3 repetitions using a load estimated to be near-maximal. 1RM attempts then commenced, with the goal of obtaining the 1RM in between 3 and 5 attempts. Three minutes of rest were allowed between attempts. The maximal weight lifted with proper form was recorded as the 1RM. After the 1RM was obtained, a 3-minute rest period was allowed before repetitions to failure (RTF) were completed using 70% of the 1RM. For all participants, the bench press was tested before the leg press in order to allow for recovery of the lower body following the mechanized squat testing.

### Metabolic and Physiological Measures

REE and substrate utilization were assessed via indirect calorimetry (TrueOne 2400. ParvoMedics, Sandy, Utah, USA). Gas and flow calibrations were performed each morning according to manufacturer specifications, and the pre-assessment procedures of Compher et al. (34) were utilized. Participants were instructed to remain motionless but awake during the assessment, which took place in a climate-controlled room with the lights dimmed. The first five minutes of each test were discarded, and the assessment continued until there was a period of 5 consecutive minutes with a coefficient of variation (CV) for REE of ≤5%. The average CV for REE in the present study was 3.2±1.1% (mean±SD).

Brachial blood pressure was measured using an automated cuff-based sphygmomanometer (HEM-907, Omron Healthcare. Kyoto, Japan). From this measurement, mean blood pressure and diastolic blood pressure were used to calibrate ensemble-averaged pressure waveforms measured at the left radial artery using applanation tonometry (SphygmoCor PVx, AtCor Medical, Itasca, Ill., USA). A general transfer function was also used to synthesize a central aortic waveform from the radial artery measurement. Wave separation analysis of the aortic pressure waveform allowed estimation of aortic pulse wave velocity (PWV), an index of arterial stiffness. Each participant remained supine for ≥10 min prior to vascular assessment. Duplicate measurements were obtained and averaged for analysis.

Blood samples collected by certified health professionals were transported via courier to a local clinical laboratory for analysis (University Medical Center Health System, Lubbock, Tex., USA). Testing was performed using standard instrumentation (Cobas 6000, Roche Diagnostics, Risch-Rotkreuz. Switzerland). Total cholesterol, triglycerides and HDL cholesterol were assessed using enzymatic colorimetric assays, and VLDL and non-HDL cholesterol were calculated. LDL cholesterol was calculated using the Martin-Hopkins equation (35). Glucose was measured using an enzymatic UV test, and insulin was assessed via electrochemiluminescence immunoassay. Results of the clinical laboratory analyses were provided to study investigators.

Each participant was familiarized with the saliva collection procedures at the baseline visit. Saliva collection took place using the passive drool method, with allows for saliva to be transferred from the mouth to a small vial according to manufacturer recommendations (36). Three saliva samples during the baseline period for assessment of the cortisol awakening response (CAR; the characteristic increase in cortisol concentrations upon waking (37)). These samples were collected at the participant's home 0, 30 and 45 minutes after waking. The importance of collecting the saliva sample exactly as instructed was strongly emphasized to research participants. Participants were provided with reminder signs to place by the bedside and were instructed to set alarms for saliva collection timepoints. Upon obtaining the sample, each participant was instructed to place the vial in the freezer until it could be transported to the laboratory. Upon delivery to the lab, each vial of saliva was stored at −80° C. until shipment to a saliva testing facility for analysis (Salimetrics LLC, Carlsbad, Calif., USA). For the analysis, samples were thawed to room temperature, vortexed, and then centrifuged for 15 minutes at approximately 3,000 RPM (1,500×g) immediately before performing the assay. Samples were tested for salivary cortisol using a high sensitivity enzyme immunoassay (Cat. No. 1-3002). Sample test volume was 25 μl of saliva per determination. The assay has a lower limit of sensitivity of 0.007 μg/dL, a standard curve range from 0.012-3.0 μg/dL, and an average intra-assay coefficient of variation of 4.60%, and an average inter-assay coefficient of variation 6.00%, which meets the manufacturers' criteria for accuracy and repeatability in Salivary Bioscience. and exceeds the applicable NIH guidelines for Enhancing Reproducibility through Rigor and Transparency.

### Questionnaires

As part of the screening procedures, participants were interviewed using a lifestyle questionnaire for determination of baseline eating and exercise habits. Participants completed follow-up lifestyle questionnaires at subsequent research visits. Additionally, participants completed the Mood and Feelings Questionnaire (38), the Pittsburgh Sleep Quality Index (39), the Three-Factor Eating Questionnaire Revised 18-item version (40) and a menstrual cycle questionnaire at each morning laboratory assessment session.

### Statistical Analysis

An a-priori power analysis was performed (G*Power, v. 3.1.9.2) using an effect size (ES) estimated from a previous investigation of TRF and RT (8). FM was specified as the primary dependent variable, and the ES used for the power analysis was the observed ES for FM reduction in TRF minus the ES for FM reduction in the control group. Using this ES (d=0.46), a a error probability of 0.05, and power of 0.8, it was estimated that 15 participants were needed to detect significant changes in fat mass. When the power analysis was performed using an ES for muscular performance improvement from the same study (d=0.25), the software estimated that 36 participants are needed to detect significant changes. Therefore, in order to promote adequate power for less sensitive measures and accounting for a 10% attrition rate, the target sample size was 40.

All data analysis occurred prior to the unblinding of study investigators and prior to receipt of urinary HMB concentrations. Data were analyzed in the intention-to-treat (ITT) framework using model-based likelihood method, meaning that the intervention effects were estimated from all participants who were randomized into the groups at baseline regardless of whether they complied with the intervention protocol (e.g., missing at follow-up assessments or drop-outs). Additional per-protocol (PP) analyses were performed by excluding participants who dropped out of the study or failed to comply with the study protocol (defined as compliance <80% with assigned eating schedule, completing fewer than 22/24 RT sessions, or <70% compliance with capsule supplements as determined by capsule counts). For both ITT and PP analyses, a linear mixed model with restricted maximum likelihood method was used to test changes in outcome variables over time across groups (i.e. TRF, TRF_{HMB} and CD). The model was established based on unstructured variance-covariance structure for the repeated measure and missing values were assumed to be missing at random. The normality of residuals assumption was tested using visual examination of Q-Q plots. If the group by time interaction effect was significant, simple effects tests were performed using one-way or repeated measures ANOVA as appropriate and Bonferroni adjustment for multiple comparisons. In the absence of statistically significant group by time interactions, main effects were examined with follow up using Sidak's pairwise comparisons. Cohen's d ES were calculated for each group by dividing the difference between baseline and week 8 (W8) values by the pooled standard deviation. A familywise alpha level of <0.05 was used for statistical significance, and all data analyses were performed using IBM SPSS v. 25 and Microsoft Excel v. 16.16.3.

### Results

### Participants

Forty participants were randomized and included in the ITT analysis, while 24 participants were included in the PP analysis. No baseline differences were present in either analysis (Table 2).

TABLE 2
Participant Characteristics.

| | PP | | | | ITT | | | |
|---|---|---|---|---|---|---|---|---|
| | CD | TRF_{HMB} | TRF | P | CD | TRF_{HMB} | TRF | P |
| | (n = 9) | (n = 7) | (n = 8) | (group) | (n = 14) | (n = 13) | (n = 13) | (group) |
| Age (y) | 22.6 ± 2.7 | 22.3 ± 3.3 | 23.3 ± 1.5 | 0.76 | 22.0 ± 2.4 | 22.3 ± 3.4 | 22.1 ± 2.1 | 0.95 |
| Body mass (kg) | 62.0 ± 8 6 | 63.7 ± 7.0 | 67.4 ± 8.1 | 0.39 | 64.6 ± 8.8 | 63.2 ± 6.1 | 63.8 ± 8.5 | 0.89 |
| Height (cm) | 170.0 ± 7.7 | 165.8 ± 5.7 | 164.0 ± 6.1 | 0.18 | 169.4 ± 7.5 | 166.0 ± 4.8 | 163.6 ± 5.9 | 0.07 |
| RT Experience (y) | 6.4 ± 3.2 | 4.8 ± 1.8 | 4.9 ± 1.8 | 0.31 | 5.4 ± 3.0 | 5.1 ± 2.1 | 5.0 ± 1.9 | 0.85 |
| Current RT (d/week) | 3.0 ± 0.5 | 3.0 ± 0.9 | 3.3 ± 0.7 | 0.57 | 2.9 ± 0.5 | 3.0 ± 0.9 | 3.3 ± 0 6 | 0.22 |

Mean ± SD: P values from one-way ANOVA.
CD: control diet: ITT: intention-to-treat: PP: per protocol: RT: resistance training: TRF: time-restricted feeding: TRF_{HMB}: time-restricted feeding plus beta-hydroxy beta-methylbutyrate supplementation

Although participants were not excluded for noncompliance in the ITT analysis, average group compliance with the assigned protocol was ≥89% for the assigned eating schedule and ≥84% for the assigned capsule supplementation based on capsule count (Supplemental Table 1). In the PP analysis, group compliance was ≥91% for the eating schedule and ≥87% for capsule supplementation. In both analyses, urinary HMB concentrations increased significantly in TRF_{HMB} from the pre-intervention period to the intervention, with no changes in TRF or CD (Supplemental Table 2).

### Nutrition and Supplementation

Prior to the intervention, there were no differences in the time of the first or last eating occasion of the day, nor the total duration of the feeding window (Supplemental Table 3). During the intervention, the time of the first eating occasion was later in TRF and TRF_{HMB} as compared to CD, while the time of the last eating occasion was later in CD. These differences resulted in a significantly longer feeding window for CD (ITT: 13.2±1.6 h/d, PP: 13.3±1.8 h/d) as compared to TRF (ITT: 7.5±0.6 h/d; PP: 7.5±0.5 h/d) or TRF_{HMB} (ITT: 7.6±0.7 h/d; PP: 7.5±0.5 h/d). Within the feeding windows, the meal frequency did not differ between groups before or during the intervention.

During the pre-intervention period, analysis of weighed diet records indicated that all groups had an average energy intake that was comparable to baseline REE (ITT: 0 to −164 kcal/d, PP: −55 to −194 kcal/d). During the intervention, energy intake increased in all groups (ITT: 23 to 194 kcal/d, PP: 90 to 250 kcal/d), with no differences between groups (Table 3).

TABLE 3
Nutrient intake.

| | | PP | | | | | | | | ITT |
|---|---|---|---|---|---|---|---|---|---|---|
| | | | Pre- | During | | Δ | P | P | P | Pre- |
| | | Group | Intervention | Intervention | Δ | (%) | (group) | (time) | (I) | Intervention |
| Energy (kcal) | | CD | 1431 ± 122 | 1681 ± 108 | 250 | 17 | 0.45 | 0.11 | 0.81 | 1384 ± 117 |
| | | TRF_{HMB} | 1352 ± 138 | 1442 ± 123 | 90 | 7 | | | | 1466 ± 111 |
| | | TRF | 1392 ± 129 | 1554 ± 115 | 162 | 12 | | | | 1430 ± 121 |
| Protein | g | CD | 68 ± 8 | 103 ± 7 | 35 | 51 | 0.36 | <0.001* | 0.27 | 67 ± 7 |
| | | TRF_{HMB} | 82 ± 10 | 98 ± 8 | 16 | 20 | | | | 77 ± 8 |
| | | TRF | 90 ± 9 | 108 ± 8 | 18 | 20 | | | | 79 ± 8 |
| | % | CD | 19 ± 2 | 26 ± 2 | 7 | 37 | 0.17 | 0.01* | 0.33 | 20 ± 2 |
| | | TRF_{HMB} | 25 ± 3 | 28 ± 2 | 3 | 12 | | | | 21 ± 2 |
| | | TRF | 27 ± 2 | 29 ± 2 | 2 | 7 | | | | 23 ± 2 |
| | g/kg | CD | 1.1 ± 0.1 | 1.7 ± 0.1 | 0.6 | 55 | 0.92 | 0.001* | 0.28 | 1.1 ± 0.1 |
| | | TRF_{HMB} | 1.3 ± 0.2 | 1.5 ± 0.1 | 0.2 | 15 | | | 0.28 | 1.2 ± 0.1 |
| | | TRF | 1.3 ± 0.2 | 1.6 ± 0.1 | 0.3 | 23 | | | | 1.2 ± 0.1 |
| Carb | g | CD | 172 ± 18 | 180 ± 18 | 8 | 5 | 0.09 | 0.58 | 1.80 | 158 ± 15 |
| | | TRF_{HMB} | 138 ± 20 | 130 ± 21 | −8 | −6 | | | | 146 ± 16 |
| | | TRF | 138 ± 19 | 157 ± 19 | 19 | 14 | | | | 167 ± 16 |
| | % | CD | 50 ± 4 | 43 ± 2 | −7 | −14 | 0.10 | 0.15 | 0.41 | 47 ± 3 |
| | | TRF_{HMB} | 41 ± 5 | 36 ± 3 | −5 | −12 | | | | 40 ± 3 |
| | | TRF | 39 ± 4 | 40 ± 2 | 1 | 3 | | | | 45 ± 3 |
| | g/kg | CD | 2.9 ± 0.3 | 2.9 ± 0.3 | 0.0 | 0 | 0.08 | 0.79 | 0.90 | 2.5 ± 0.3 |
| | | TRF_{HMB} | 2.1 ± 0.3 | 2.1 ± 0.4 | 0.0 | 0 | | | | 2.3 ± 0.3 |
| | | TRF | 2.1 ± 0.3 | 2.3 ± 0.3 | 0.2 | 10 | | | | 2.7 ± 0.5 |
| Fat | g | CD | 48 ± 8 | 57 ± 5 | 9 | 19 | 0.93 | 0.42 | 0.71 | 51 ± 10 |
| | | TRF_{HMB} | 54 ± 9 | 58 ± 6 | 4 | 7 | | | | 75 ± 11 |
| | | TRF | 55 ± 9 | 55 ± 5 | 0 | 0 | | | | 52 ± 11 |
| | % | CD | 31 ± 3 | 31 ± 2 | 0 | 0 | 0.46 | 0.90 | 0.59 | 34 ± 3 |
| | | TRF_{HMB} | 34 ± 4 | 36 ± 2 | 2 | 6 | | | | 39 ± 3 |
| | | TRF | 35 ± 3 | 32 ± 2 | −3 | −9 | | | | 32 ± 3 |
| | g/kg | CD | 0.8 ± 0.1 | 0.9 ± 0.1 | 0.1 | 13 | 0.95 | 0.46 | 0.69 | 0.8 ± 0.2 |
| | | TRF_{HMB} | 0.9 ± 0.2 | 0.9 ± 0.1 | 0.0 | 0 | | | | 1.2 ± 0 2 |
| | | TRF | 0.8 ± 0.1 | 0.8 ± 0.1 | 0.0 | 0 | | | | 0.8 ± 0.2 |

| | | | ITT | | | | | |
|---|---|---|---|---|---|---|---|---|
| | | PP | During | | Δ | P | P | P |
| | | Group | Interventionn | Δ | (%) | (group) | (time) | (I) |
| Energy (kcal) | | CD | 1570 ± 111 | 186 | 13 | 0.91 | 0.010* | 0.62 |
| | | TRF_{HMB} | 1489 ± 112 | 23 | 2 | | | |
| | | TRF | 1624 ± 107 | 194 | 14 | | | |
| Protein | g | CD | 98 ± 7 | 31 | 46 | 0.49 | <0.0001* | 0.87 |
| | | TRF_{HMB} | 102 ± 7 | 25 | 32 | | | |
| | | TRF | 105 ± 6 | 26 | 33 | | | |
| | % | CD | 27 ± 2 | 7 | 35 | 0.67 | <0.001* | 0.48 |
| | | TRF_{HMB} | 28 ± 2 | 7 | 33 | | | |
| | | TRF | 27 ± 2 | 4 | 17 | | | |
| | g/kg | CD | 1.6 ± 0.1 | 0.5 | 45 | 0.58 | <0.0001* | 0.82 |
| | | TRF_{HMB} | 1.6 ± 0.1 | 0.4 | 33 | | | |
| | | TRF | 1.6 ± 0.1 | 0.4 | 33 | | | |
| Carb | g | CD | 165 ± 17 | 7 | 4 | 0.58 | 0.90 | 0.83 |
| | | TRF_{HMB} | 145 ± 17 | −1 | −1 | | | |
| | | TRF | 157 ± 16 | −10 | −6 | | | |
| | % | CD | 42 ± 2 | −5 | −11 | 0.24 | 0.045* | 0.58 |
| | | TRF_{HMB} | 39 ± 2 | −1 | −3 | | | |
| | | TRF | 39 ± 2 | −6 | −13 | | | |
| | g/kg | CD | 2.6 ± 0.3 | 0.1 | 4 | 0.59 | 0.82 | 0.81 |
| | | TRF_{HMB} | 2.3 ± 0.3 | 0.0 | 0 | | | |
| | | TRF | 2.5 ± 0.3 | −0.2 | −7 | | | |
| Fat | g | CD | 54 ± 6 | 3 | 6 | 0.46 | 0.74 | 0.12 |
| | | TRF_{HMB} | 53 ± 6 | −22 | −29 | | | |
| | | TRF | 64 ± 5 | 12 | 23 | | | |
| | % | CD | 32 ± 2 | −2 | −6 | 0.34 | 0.24 | 0.20 |
| | | TRF_{HMB} | 32 ± 3 | −7 | −18 | | | |
| | | TRF | 34 ± 2 | 2 | 6 | | | |
| | g/kg | CD | 0.9 ± 0.1 | 0.1 | 13 | 0.46 | 0.80 | 0.11 |
| | | TRF_{HMB} | 0.8 ± 0.1 | −0.4 | −33 | | | |
| | | TRF | 1.0 ± 0.1 | 0.2 | 25 | | | |

Mean ± SE; P values from mixed model analysis;
*Significant change in all groups combined (time main effect)
CD: control diet; I: group by time interaction; ITT: intention-to-treat; PP: per protocol; TRF: time-restricted feeding; TRF_{HMB}: time-restricted feeding plus beta-hydroxy beta-methylbutyrate supplementation

The magnitude of increase in energy intake approximated the average daily calories consumed from the provided whey protein supplements (˜200 to 250 kcal/d). Despite this increase in energy intake, daily caloric consumption remained near baseline REE (ITT: +22 to 75 kcal/d, PP: −32 to +195 kcal/d) and W8 REE (ITT: +8 to 93 kcal/d. PP: −77 to +240 kcal/d). Protein intake in all groups increased from the pre-intervention period to the intervention, with average intakes of 1.5 to 1.7 g/kg/d during the intervention. Carbohydrate and fat intake generally did not change during the intervention.

### Resistance Training Program and Physical Activity Monitoring

There were no differences between groups for upper- or lower-body session volume or total volume in either analysis (Supplemental Table 4). In all groups, volume increased from the first half of the intervention to the second half of the intervention, with the magnitude of increase in group session volume ranging from 15 to 27%. During the intervention, group step counts ranged from 7,354 to 8,830 steps/day, with no significant differences between groups or across time (Supplemental Table 5). Group by time interactions were present for PAEE, sedentary time and light-intensity PA. Differences between groups were present in the pre-intervention period for sedentary time and light-intensity PA, but not during the early or late intervention periods. Furthermore, no statistically significant differences between time points within a group were observed, with the exception of higher sedentary time observed in the TRF group during the early intervention as compared to pre-intervention in the ITT analysis.

### Body Composition

In the PP analysis, FFM increased by 1.0 to 1.4 kg in all groups without significant differences between groups (Table 4). However, fat free mass gain was numerically greater in the TRF+HMB group over CD or TRF alone (1.4 kg in TRF+HMB vs. 1.1 in CD and 1.0 in TRF) and had a larger effect size (0.32 v. 0.25 and 0.23).

TABLE 4
Body Composition.

| | PP | | | | | | | | | |
|---|---|---|---|---|---|---|---|---|---|---|
| | | | | | | Δ | ES | P | P | P |
| | Group | Baseline | Week 4 | Week 8 | Δ | (%) | (d) | (group) | (time) | (I) |
| BM | CD | 62.0 ± 2.7 | 63.3 ± 2.7 | 63.5 ± 2.6 | 1.5 | 2 | 0.19 | 0.49 | 0.10 | 0.19 |
| (kg) | TRF_{HMB} | 63.7 ± 3.0 | 64.2 ± 3.1 | 63.7 ± 3.0 | 0.0 | 0 | 0.00 | | | |
| | TRF | 67.4 ± 2.3 | 67.3 ± 2.9 | 67.6 ± 2.8 | 0.2 | 0 | 0.03 | | | |
| FM | CD | 17.7 ± 2.1 | 17.6 ± 2.2 | 18.1 ± 2.1 | 0.4 | 2 | 0.06 | 0.93 | 0.004* | 0.03* |
| (kg) | TRF_{HMB} | 18.7 ± 2.3 | 17.5 ± 2.5 | 17.3 ± 2.3^{b} | −1.4 | −7 | −0.23 | | | |
| | TRF | 19.7 ± 2.2 | 18.0 ± 2.3^{b} | 18.9 ± 2.2 | −0.8 | −4 | −0.13 | | | |
| FFM | CD | 44.3 ± 1.5 | 45.6 ± 1.6 | 45.4 ± 1.4 | 1.1 | 2 | 0.25 | 0.28 | <0.0001*^{a} | 0.92 |
| (kg) | TRF_{HMB} | 45.0 ± 1.7 | 46.8 ± 1.8 | 46.4 ± 1.6 | 1.4 | 3 | 0.32 | | | |
| | TRF | 47.7 ± 1.6 | 49.4 ± 1.6 | 48.7 ± 1.5 | 1.0 | 2 | 0.23 | | | |
| BF | CD | 28.1 ± 2.2 | 27.3 ± 2.4 | 28.0 ± 2.3 | −0.1 | 0 | −0.01 | 0.99 | 0.0001* | 0.048* |
| % | TRF_{HMB} | 29.1 ± 2.5 | 27.0 ± 2.7^{b} | 26.8 ± 2.6^{b} | −2.3 | −8 | −0.34 | | | |
| | TRF | 28.7 ± 2.4 | 26.0 ± 2.6^{b} | 27.3 ± 2.4 | −1.4 | −5 | −0.21 | | | |
| MT_{EF} | CD | 2.77 ± 0.10 | — | 2.90 ± 0.09 | 0.13 | 5 | 0.46 | 0.17 | <0.001*^{d} | 0.58 |
| (cm) | TRF_{HMB} | 2.73 ± 0.10 | — | 2.97 ± 0.10 | 0.24 | 9 | 0.86 | | | |
| | TRF | 2.98 ± 0.10 | — | 3.14 ± 0.10 | 0.16 | 5 | 0.57 | | | |
| MT_{KE} | CD | 3.92 ± 0.20 | — | 4.25 ± 0.17 | 0.33 | 8 | 0.59 | 0.001*^{e} | 0.0001*^{d} | 0.43 |
| (cm) | TRF_{HMB} | 4.27 ± 0.22 | — | 4.44 ± 0.20 | 0.17 | 4 | 0.31 | | | |
| | TRF | 5.04 ± 0.21 | — | 5.40 ± 0.18 | 0.36 | 7 | 0.65 | | | |

| | | ITT | | | | | | | | |
|---|---|---|---|---|---|---|---|---|---|---|
| | PP | | | | | Δ | ES | P | P | P |
| | Group | Baseline | Week 4 | Week 8 | Δ | (%) | (d) | (group) | (time) | (I) |
| BM | CD | 64.7 ± 2.1 | 65.7 ± 2.1 | 65.8 ± 2.1 | 1.1 | 2 | 0.14 | 0.84 | 0.01*^{a} | 0.24 |
| (kg) | TRF_{HMB} | 63.2 ± 2.2 | 63.9 ± 2.2 | 63.8 ± 2.1 | 0.6 | 1 | 0.08 | | | |
| | TRF | 63.8 ± 2.2 | 63 9 ± 2 2 | 64.4 ± 2.1 | 0.6 | 1 | 0.08 | | | |
| FM | CD | 19.2 ± 1.4 | 19.4 ± 1.5 | 19.6 ± 1.4 | 0.4 | 2 | 0.08 | 0.66 | 0.02*^{c} | 0.08 |
| (kg) | TRF_{HMB} | 18.2 ± 1.5 | 17.5 ± 1.5 | 17.5 ± 1.5 | −0.7 | −4 | −0.13 | | | |
| | TRF | 18.4 ± 1.5 | 17.2 ± 1.5 | 18.0 ± 1.4 | −0.4 | −2 | −0.08 | | | |
| FFM | CD | 45.4 ± 1.3 | 46.3 ± 1.3 | 46.3 ± 1.2 | 0.9 | 2 | 0.19 | 0.99 | <0.0001*^{a} | 0.81 |
| (kg) | TRF_{HMB} | 45.0 ± 1.3 | 46.5 ± 1.4 | 46.2 ± 1.3 | 1.2 | 3 | 0.26 | | | |
| | TRF | 45.5 ± 1.3 | 46.7 ± 1.4 | 46.4 ± 1.2 | 0.9 | 2 | 0.20 | | | |
| BF | CD | 29.3 ± 1.5 | 29.0 ± 1.6 | 29.4 ± 1.5 | 0.1 | 0 | 0.02 | 0.69 | 0.002*^{a} | 0.14 |
| % | TRF_{HMB} | 28.7 ± 1.5 | 27.1 ± 1.7 | 27.3 ± 1.6 | −1.4 | −5 | −0.25 | | | |
| | TRF | 28.4 ± 1.5 | 26.6 ± 1.7 | 27.6 ± 1.6 | −0.8 | −3 | −0.14 | | | |
| MT_{EF} | CD | 2.84 ± 0.09 | — | 2.96 ± 0.09 | 0.12 | 4 | 0.36 | 0.76 | 0.001*^{d} | 0.41 |
| (cm) | TRF_{HMB} | 2.74 ± 0.09 | — | 2.96 ± 0.09 | 0.22 | 8 | 0.68 | | | |
| | TRF | 2.88 ± 0.09 | — | 2.98 ± 0.08 | 0.10 | 3 | 0.33 | | | |
| MT_{KE} | CD | 4.07 ± 0.16 | — | 4.38 ± 0.16 | 0.31 | 8 | 0.52 | 0.009*^{e} | <0.0001*^{d} | 0.48 |
| (cm) | TRF_{HMB} | 4.14 ± 0.16 | — | 4.33 ± 0.16 | 0.19 | 5 | 0.33 | | | |
| | TRF | 4.67 ± 0.16 | — | 5.0 1± 0.15 | 0.34 | 7 | 0.61 | | | |

Mean ± SE; P values from mixed model analysis;
*Statistically significant (p < 0.05);
^{a}Significantly different than baseline at W 4 and W 8 in all groups combined;
^{b}Significantly different than baseline value in specified group;
^{c}Significantly different than baseline at W 4 in all groups combined;
^{d}Significantly different than baseline value in all groups combined;
^{e}Baseline value higher in TRF than CD
BF %: 4-component model body fat percentage; BM: body mass; CD: control diet; ES: effect size; FM: 4-component model fat mass; FFM: 4-component model fat-free mass; I: group by time interaction; ITT: intention-to-treat; MT_{EF}: ultrasound muscle thickness of elbow flexors; MT_{KE}: ultrasound muscle thickness of knee extensors; PP: per protocol; TRF: time-restricted feeding; TRF_{HMB}: time-restricted feeding plus beta-hydroxy beta-methylbutyrate supplementation

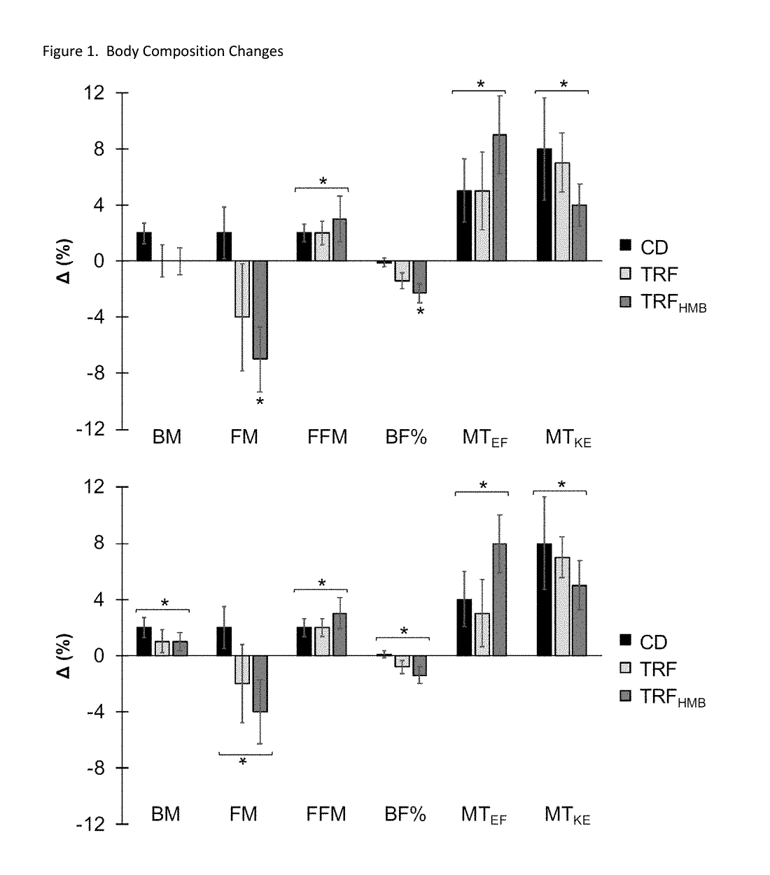

Fat mass did not change in CD, but significant reductions were observed in TRF and TRF_{HMB} (FIG. 1). In FIG. 1, percent changes (mean±SEM) are displayed as differences between baseline and final values relative to baseline values for each variable. The upper panel displays results for per protocol (PP) analysis and the bottom panel displays results for intention-to-treat (ITT) analysis. Total body composition was estimated using a 4-component model, while muscle thickness was assessed via ultrasonography. Asterisks with brackets indicate significant changes in all groups (i.e. time main effects), with non-significant differences between groups, based on mixed model analysis. Asterisks above only one column indicate a change in only the specified group (i.e. significant group by time interaction in mixed model analysis with follow up tests).

Although FM was significantly lower than baseline at week 4 (W4) in TRF, FM at W8 did not significantly differ from baseline. In contrast. FM in TRF_{HMB} was lower at W8 than baseline. No changes in BF % were observed in CD, and the reduction in BF % was statistically significant in TRF_{HMB}, but not TRF, at W8. Time main effects were present for MT_{EF} and MT_{KE}, indicating increases in all groups. In the IT analysis, FFM increased by 0.9 to 1.2 kg in all groups without significant differences between groups. In contrast to the PP analysis, the group by time interaction was not statistically significant for FM or BF %, although time main effects indicated decreases in FM and BF % in all groups combined. Although not statistically significant, the magnitude of increases in muscle thickness appeared potentially disparate between groups for the upper and lower body in both analyses.

### Muscular Performance

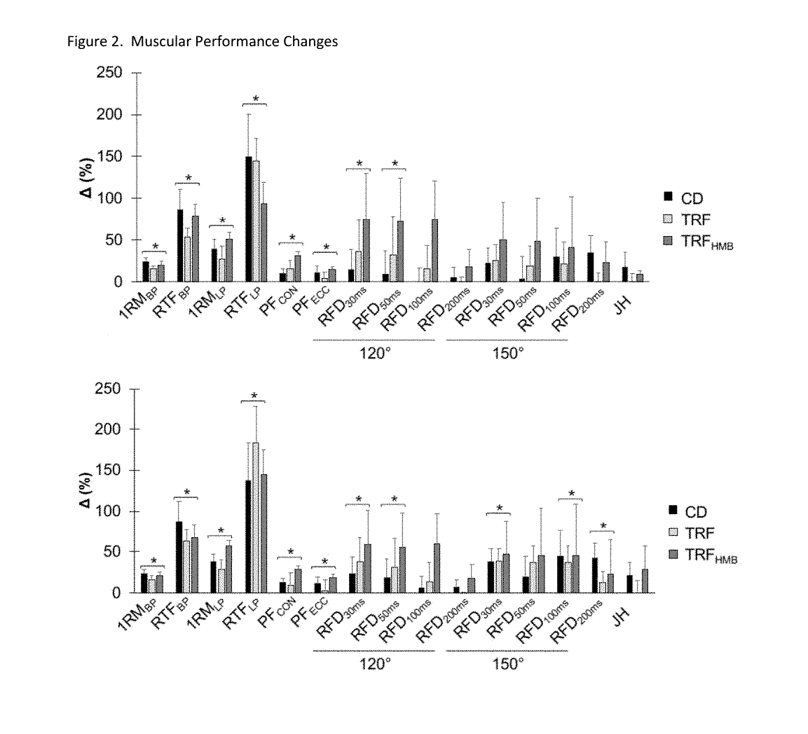

Maximal strength and muscular endurance improved in all groups without statistically significant differences between groups (FIG. 2; Table 5). Muscular performance improved without significant differences between groups, although average effect sizes for tests of lower body force generation favored TRF_{HMB} (d=0.6-0.7) as compared to TRF or CD (d=0.3-0.4).

TABLE 5
Muscular Performance

| | PP | | | | | | | | | |
|---|---|---|---|---|---|---|---|---|---|---|
| | | | | | | Δ | ES | P | P | P |
| | Group | Baseline | Week 4 | Week 8 | Δ | (%) | (d) | (group) | (time) | (I) |
| 1RM_{BP} | CD | 38 ± 4 | 42 ± 3 | 47 ± 3 | 9 | 24 | 0.85 | 0.18 | <0.0001^{a}* | 0.73 |
| (kg) | TRF_{HMB} | 40 ± 4 | 43 ± 4 | 48 ± 4 | 8 | 20 | 0.76 | | | |
| | TRF | 48 ± 4 | 50 ± 3 | 55 ± 3 | 7 | 15 | 0.70 | | | |
| RTF_{BP} | CD | 15 ± 1 | 22 ± 2 | 28 ± 2 | 13 | 87 | 2.74 | 0.22 | <0.0001^{a}* | 0.30 |
| (reps) | TRF_{HMB} | 14 ± 1 | 20 ± 2 | 25 ± 3 | 11 | 79 | 1.86 | | | |
| | TRF | 15 ± 1 | 16 ± 2 | 23 ± 2 | 8 | 53 | 1.79 | | | |
| 1RM_{LP} | CD | 130 ± 10 | — | 181 ± 17 | 51 | 39 | 1.22 | 0.11 | <0.0001^{a}* | 0.30 |
| (kg) | TRF_{HMB} | 146 ± 11 | — | 220 ± 19 | 74 | 51 | 1.80 | | | |
| | TRF | 172 ± 10 | — | 218 ± 19 | 46 | 27 | 1.07 | | | |
| RTF_{LP} | CD | 14 ± 2 | — | 35 ± 5 | 21 | 150 | 1.84 | 0.50 | <0.0001^{a}* | 0.77 |
| (reps) | TRF_{HMB} | 16 ± 2 | — | 31 ± 6 | 15 | 94 | 1.27 | | | |
| | TRF | 11 ± 2 | — | 27 ± 6 | 16 | 145 | 1.26 | | | |
| PF_{CON} | CD | 1104 ± 96 | 1133 ± 117 | 1214 ± 136 | 110 | 10 | 0.31 | 0.41 | 0.001*^{b} | 0.43 |
| (N) | TRF_{HMB} | 1082 ± 108 | 1247 ± 133 | 1418 ± 154 | 336 | 31 | 0.95 | | | |
| | TRF | 1266 ± 101 | 1387 ± 125 | 1458 ± 146 | 192 | 15 | 0.54 | | | |
| PF_{ECC} | CD | 1275 ± 139 | 1334 ± 120 | 1415 ± 118 | 140 | 11 | 0.36 | 0.73 | 0.04*^{c} | 0.82 |
| (N) | TRF_{HMB} | 1291 ± 158 | 1311 ± 136 | 1475 ± 133 | 184 | 14 | 0.48 | | | |
| | TRF | 1447 ± 148 | 1466 ± 127 | 1504 ± 127 | 57 | 4 | 0.15 | | | |
| | | ITT | | | | | | | | |
| | PP | | | | | Δ | ES | P | P | P |
| | Group | Baseline | Week 4 | Week 8 | Δ | (%) | (d) | (group) | (time) | (I) |
| 1RM_{BP} | CD | 38.1 ± 2.6 | 42.4 ± 2.3 | 47.2 ± 2.4 | 9 | 24 | 0.94 | 0.40 | <0.0001^{a}* | 0.64 |
| (kg) | TRF_{HMB} | 39.0 ± 2.7 | 42.3 ± 2.4 | 47.3 ± 2.5 | 8 | 21 | 0 86 | | | |
| | TRF | 43.5 ± 2.7 | 45.7 ± 2.4 | 51.0 ± 2.4 | 8 | 17 | 0.79 | | | |
| RTF_{BP} | CD | 14.7 ± 0.9 | 21.7 ± 1.5 | 27.7 ± 1.8 | 13 | 88 | 2.37 | 0.47 | <0.0001^{b}* | 0.37 |
| (reps) | TRF_{HMB} | 14.8 ± 0.9 | 20.0 ± 1.6 | 24.9 ± 1.8 | 10 | 68 | 1.91 | | | |
| | TRF | 15.2 ± 0.9 | 18.4 ± 1.6 | 24.7 ± 1.7 | 10 | 63 | 1.83 | | | |
| 1RM_{LP} | CD | 134.3 ± 8.1 | — | 185.7 ± 15.2 | 51 | 38 | 1.10 | 0.49 | <0.0001^{c}* | 0.17 |
| (kg) | TRF_{HMB} | 130.4 ± 8.4 | — | 205.2 ± 15.0 | 75 | 57 | 1.65 | | | |
| | TRF | 155.9 ± 8.8 | — | 200.5 ± 14.8 | 45 | 29 | 0.98 | | | |
| RTF_{LP} | CD | 14.8 ± 1.5 | — | 35.2 ± 5.6 | 20 | 138 | 1.29 | 0.43 | <0.0001^{c}* | 0.95 |
| (reps) | TRF_{HMB} | 15.5 ± 1.5 | — | 38.0 ± 5.4 | 23 | 145 | 1.52 | | | |
| | TRF | 11.2 ± 1.6 | — | 31.8 ± 5.1 | 21 | 184 | 1.46 | | | |
| PF_{CON} | CD | 1090 ± 74 | 1155 ± 87 | 1234 ± 109 | 144 | 13 | 0.40 | 0.79 | 0.001*^{a} | 0.52 |
| (N) | TRF_{HMB} | 1029 ± 76 | 1195 ± 92 | 1324 ± 112 | 295 | 29 | 0.83 | | | |
| | TRF | 1175 ± 76 | 1249 ± 92 | 1296 ± 111 | 121 | 10 | 0.34 | | | |
| PF_{ECC} | CD | 1244 ± 101 | 1316 ± 89 | 1396 ± 96 | 152 | 12 | 0.40 | 0.84 | 0.009*^{c} | 0.58 |
| (N) | TRF_{HMB} | 1184 ± 105 | 1301 ± 93 | 1407 ± 95 | 223 | 19 | 0.60 | | | |
| | TRF | 1339 ± 105 | 1384 ± 93 | 1383 ± 93 | 44 | 3 | 0.12 | | | |

Mean ± SE; P values from mixed model analysis
^{a}Significantly different between each time point in all groups combined;
^{b}Significantly different than baseline and W 4 at W 8;
^{c}Significantly different than baseline at W 8 PP. No baseline differences were present between groups with the exception of greater 1RM_{LP} in TRF as compared to CD (p = 0.02) in the PP analysis.
1-RM_{BP}: 1-repetition maximum on bench press exercise; 1-RMLP: 1-repetition maximum on leg press exercise; CD: control diet; ES: effect size; I: group by time interaction; ITT: intention-to-treat; PF_{CON}: concentric peak force; PF_{ECC}: eccentric peak force; PP: per protocol; RIF _{BP}: repetitions to failure on bench press exercise using 70% of baseline 1-RM. RTF_{LP}: repetitions to failure on leg press exercise using 70% of baseline 1-RM; TRF: time-restricted feeding; TRF_{HMB}: time-restricted feeding plus beta-hydroxy beta-methylbutyrate supplementation.

In FIG. 2, percent changes (mean+SEM) are displayed as differences between baseline and final values relative to baseline values for each variable. The upper panel displays results for per protocol (PP) analysis and the bottom panel displays results for intention-to-treat (ITT) analysis. Asterisks with brackets indicate significant changes in all groups (i.e. time main effects), with non-significant differences between groups, based on mixed model analysis. Maximal strength (1RM) and repetitions to failure (RTF) were obtained for the leg press and bench press exercises, peak forces (PF) were obtained from isokinetic squat testing, rate of force development (RFD) was obtained from isometric squat testing, and jump height (JH) was calculated using force platforms. Durations over which RFD values were calculated are shown in subscripts.

Several RFD variables were also improved in all groups, particularly in the ITT analysis (Supplemental Table 6). A trend (p=0.06) for a time main effect for increased jump height was observed in the ITT analysis, although the ES in CD (d=0.63) and TRF_{HMB} (d=0.65) appeared larger than TRF (d=0.00) (Supplemental Table 7).

### Metabolic and Physiological Variables

No significant changes in REE or RQ were observed in any group (Supplemental Table 8). In the CD and TRF groups, non-significant reductions in REE of 45 to 71 kcal/d (d=−0.29 to −0.42) were observed, while REE was 15 to 47 kcal/d higher than baseline in the TRF_{HMB} (d=0.09 to 0.30). Resting metabolic rate increase in the TRF+HMB group (+47 kcal/d; 3%) while decreasing in the CD (−45 kcal/d; −3%) and TRF (−63 kcal/d; −4%) groups. Blood markers were generally unchanged by the study intervention, although a significant time main effect for increased LDL was observed in the PP analysis (Supplemental Table 9). No significant changes in vascular assessments, cortisol awakening response or average cortisol concentrations were observed (Supplemental Tables 10 & 11).

### Questionnaires

Overall, no major side effects or adverse events occurred during the study. At W4, 84% of participants reported no side effects. Reported side effects included both suppressed appetite (n=1) and increased appetite with associated irritability (n=1) in TRF, morning fatigue in TRF_{HMB} (n=1), nausea in CD (n=1) and bloated stomach in CD and TRF_{HMB} (n=1 each). At W8, 90% of participants reported no side effects. Reported side effects included suppressed appetite (n=1) in TRF and bloated stomach in both TRF and TRF_{HMB} (n=1 each).

No differences between groups were observed for questionnaire responses. A time main effect indicated improvement in scores for the Mood and Feelings Questionnaire at W4 and W8 compared to baseline in all groups (Supplemental Table 12). In the ITT analysis, the uncontrolled eating score of the Three Factor Eating Questionnaire was reduced across time in all groups, with a trend for the same effect in the PP analysis. The proportion of participants with regularly-occurring menstrual cycles in each group ranged from 57 to 78% in the PP analysis and from 69 to 79% in the ITT analysis (Supplemental Table 13).

### Discussion

The present investigation is the first trial of IF plus RT in female participants. The purpose of the trial was to compare the effects of TRF, with or without HMB supplementation during fasting periods, to a control diet requiring breakfast consumption during progressive RT.

In the present investigation, adherence to TRF resulted in loss of FM without hindering FFM accretion, skeletal muscle hypertrophy or improvements in muscular performance. In the PP analysis, FM decreased in TRF and TRF_{HMB}. In the ITT analysis, the magnitude of effects was lessened as expected. Despite the resultant lack of statistical significance between groups for FM and BF %, the same trends were observed as in the PP analysis. While improvements in muscular performance did not vary significantly between groups, the magnitude of improvement for measures related to rapid force generation in the lower body, including 1RM_{LP}, PF_{CON}, PF_{ECC}, and RFD, are disparate between groups. For these measures, the average ES in TRF_{HMB} was 0.6 to 0.7 as compared to 0.3 to 0.4 in both CD and TRF.

In contrast to metrics of rapid force generation, the magnitude of improvements in muscular endurance (i.e. RTF_{LP} and RTF_{BP}) may have favored the dietary pattern including a longer feeding window (i.e. CD) in the PP analysis only, with an average ES of 2.3 in CD, but 1.5 in TRF and TRF_{HMB}.

Dietary advice provided in the present investigation was minimal. Specifically, each participant met briefly (<10 min) to discuss the assigned eating schedule and protein consumption target with the primary investigator at the time of group assignment. Two additional follow up visits of similar duration allowed for discussion of the results of weighed diet records. Although the shortcomings of self-reported dietary intake are well-established and resultant nutrient intake estimates should be viewed cautiously (50, 51), weighed diet records revealed no significant differences between groups for energy or macronutrient intake. As estimated energy intake was typically below the target intake, the primary dietary feedback was to achieve a high protein intake through consumption of protein-containing foods and the provided supplement. In all groups, average protein intake increased from 1.1 to 1.3 g/kg/d in the pre-intervention period to 1.5 to 1.7 g/kg/d during the study intervention, a range consistent with optimal intake for muscular adaptations (9, 10).

It has been recognized that longitudinal data are needed to elucidate the impact of the daily distribution of protein intake on adaptations to RT (9). As IF necessitates prolonged periods without stimulation of muscle protein synthesis and suppression of muscle protein breakdown via dietary amino acids (13), it represents an opportunity to investigate this question. The present investigation reveals no detrimental effects on RT adaptations of limiting all protein and other nutrient intake to ˜7.5 h/d, as compared to ˜13.5 h/d. In the context of IF, it has also been questioned whether implementation of modified fasting periods to allow ingestion of selected amino acids or their metabolites may be beneficial for lean mass maintenance or accretion, particularly in active individuals (14). The present investigation is the first trial to directly examine this question and reveals the benefits of HMB supplementation for FM reduction and of lower body muscular performance.

Supplemental HMB during fasting periods of a TRF program enhances fat loss as compared to TRF alone and benefits lower body muscular performance.

### Experimental Example 2

The amount of fat loss that occurs with β-hydroxy-β-methylbutyrate (HMB) supplementation can be increased when combined with intermittent fasting. In this example, it is demonstrated that HMB supplementation with intermittent fasting results in greater fat loss than HMB supplementation alone.

In Example 1, active females (n=7, 22±3.3 y, 63.7±7.0 kg) were randomized to a time-restricted feeding plus 3 g/d Calcium-HMB (TRFHMB). TRFHMB group consumed all calories in ˜8 h/d. TRFHMB group completed 8 weeks of supervised resistance training. Body composition was assessed at baseline, and 4 and 8 weeks using a modified 4-component (4C) model^{1,2} produced from dual-energy x-ray absorptiometry (DXA) and bioimpedance spectroscopy (BIS) data. DXA scans were performed on a Lunar Prodigy scanner (General Electric. Boston, Mass. USA) with enCORE software (v. 16.2).

In an earlier study described in Panton et al. (54) trained and untrained females (n=18, 27±2.1 y, 62.3±2.2 kg) were randomized to 3 g/d Calcium-HMB without intermitted fasting. The HMB only group completed 4 weeks of supervised resistance training and trained three times per week. Body composition was measured before and after the 4 weeks of training using underwater weighing procedures (55). Percent body fat (BF %) was estimated from the Siri equation⁵.

In the TRFHMB group, BF % decreased (p<0.05) from 29.1±2.5 to 27.0±2.7% in 4 weeks. The 4-week Δ-change was −2.1% with an effect size of d=−0.31. This fat loss effect was maintained through 8 weeks. In the HMB only group, BF % decreased nonsignificantly from 23.7±1.1 to 23.0±1.2% in 4 weeks. The 4-week Δ-change was −0.7% with an effect size of d=−0.15. The absolute effect size was 2-fold greater with TRFHMB and indicates a stronger effect for BF % loss when HMB supplementation is combined with intermittent fasting.

In conclusion, these data surprisingly support the use of HMB supplementation combined with intermittent fasting to accelerate body fat loss compared to supplementation with HMB alone.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

### REFERENCES

1. Tinsley G, Bounty P. Effects of intermittent fasting on body composition and clinical health markers in humans. Nutrition Reviews 2015; 73. doi: 10.1093/nutrit/nuv041.
2. Anton S D, Moehl K, Donahoo W T, Marosi K, Lee S A, Mainous A G, 3rd, Leeuwenburgh C, Mattson M P. Flipping the Metabolic Switch: Understanding and Applying the Health Benefits of Fasting. Obesity (Silver Spring) 2017. doi: 10.1002/oby.22065.
3. Harris L, Hamilton S, Azevedo L B, Olajide J, De Brun C, Waller G, Whittaker V, Sharp T, Lean M, Hankey C, et al. Intermittent fasting interventions for treatment of overweight and obesity in adults: a systematic review and meta-analysis. JBI database of systematic reviews and implementation reports 2018; 16(2):507-47. doi: 10.11124/jbisrir-2016-003248.
4. Seimon R V, Roekenes J A, Zibellini J, Zhu B, Gibson A A, Hills A P, Wood R E, King N A, Byrne N M, Sainsbury A. Do intermittent diets provide physiological benefits over continuous diets for weight loss? A systematic review of clinical trials. Molecular and Cellular Endocrinology 2015; 418 Pt 2:153-72. doi: 10.1016/j.mce.2015.09.014.
5. Davis C S, Clarke R E, Coulter S N, Rounsefell K N, Walker R E, Rauch C E, Huggins C E, Ryan L. Intermittent energy restriction and weight loss: a systematic review. Eur J Clin Nutr 2016; 70(3):292-9. doi: 10.1038/ejcn.2015.195.
6. Bhutani S, Klempel M C, Kroeger C M, Trepanowski J F, Varady K A. Alternate day fasting and endurance exercise combine to reduce body weight and favorably alter plasma lipids in obese humans. Obesity (Silver Spring, Md.) 2013; 21:1370-9. doi: 10.1002/oby.20353.
7. Tinsley G M, Forsse J S, Butler N K, Paoli A, Bane A A, La Bounty P M, Morgan G B, Grandjean P W. Time-restricted feeding in young men performing resistance training: A randomized controlled trial. European Journal of Sport Science 2017; 17(2):200-7. doi: 10.1080/17461391.2016.1223173.
8. Moro T, Tinsley G, Bianco A, Marcolin G, Pacelli Q F, Battaglia G, Palma A, Gentil P, Neri M, Paoli A. Effects of eight weeks of time-restricted feeding (16/8) on basal metabolism, maximal strength, body composition, inflammation, and cardiovascular risk factors in resistance-trained males. Journal of Translational Medicine 2016; 14:290. doi: 10.1186/s12967-016-1044-0.
9. Jäger R, Kerksick C M, Campbell B I, Cribb P J, Wells S D, Skwiat T M, Purpura M, Ziegenfuss T N, Ferrando A A, Arent S M, et al. International Society of Sports Nutrition Position Stand: protein and exercise. Journal of the International Society of Sports Nutrition 2017; 14(1):20. doi: 10.1186/s12970-017-0177-8.
10. Phillips S M. Dietary protein requirements and adaptive advantages in athletes. Br J Nutr 2012; 108 Suppl 2:S158-67. doi: 10.1017/s0007114512002516.
11. Heilbronn L K, Civitarese A E, Bogacka I, Smith S R, Hulver M, Ravussin E. Glucose tolerance and skeletal muscle gene expression in response to alternate day fasting. Obesity research 2005; 13:574-81. doi: 10.1038/oby.2005.61.
12. Heilbronn L, Smith S, Martin C, Anton S, Ravussin E. Alternate-day fasting in nonobese subjects: effects on body weight, body composition, and energy metabolism. Am J Clin Nutr 2005; 81.
13. McGlory C, Vliet S, Stokes T, Mittendorfer B, Phillips S M. The impact of exercise and nutrition on the regulation of skeletal muscle mass. The Journal of Physiology; 0(0). doi: doi:10.1113/JP275443.
14. Tinsley G M, Givan A H, Graybeal A J, Villarreal M I, Cross A G. β-Hydroxy β-methylbutyrate free acid alters cortisol responses, but not myofibrillar proteolysis, during a 24-h fast. British Journal of Nutrition 2018; 119(5):517-26. doi: 10.1017/50007114517003907.
15. Graybeal A J, Moore M L, Cruz M R, Tinsley G M. Body Composition Assessment in Male and Female Bodybuilders: A 4-Compartment Model Comparison of Dual-Energy X-Ray Absorptiometry and Impedance-Based Devices. The Journal of Strength & Conditioning Research 2018; Published Ahead of Print. doi: 10.1519/jsc.0000000000002831.
16. Troiano R P, Berrigan D, Dodd K W, Masse L C, Tilert T, McDowell M. Physical activity in the United States measured by accelerometer. Med Sci Sports Exerc 2008; 40(1):181-8. doi: 10.1249/mss.0b013e31815a51b3.
17. Freedson P S, Melanson E, Sirard J. Calibration of the Computer Science and Applications, Inc. accelerometer. Med Sci Sports Exerc 1998; 30(5):777-81.
18. Williams R. Kcal estimates from activity counts using the Potential Energy Method. CSA, Inc 1998; ActiGraph 49.
19. Willett W C, Howe G R, Kushi L H. Adjustment for total energy intake in epidemiologic studies. Am J Clin Nutr 1997; 65(4 Suppl):1220S-8S; discussion 95-31S. doi: 10.1093/ajcn/65.4.1220S.
20. Wilson J P, Strauss B J, Fan B, Duewer F W, Shepherd J A. Improved 4-compartment body-composition model for a clinically accessible measure of total body protein. The American Journal of Clinical Nutrition 2013; 97:497-504. doi: 10.3945/ajcn.112.048074.
21. Ng B K, Liu Y E, Wang W, Kelly T L, Wilson K E, Schoeller D A, Heymsfield S B, Shepherd J A. Validation of rapid 4-component body composition assessment with the use of dual-energy X-ray absorptiometry and bioelectrical impedance analysis. The American Journal of Clinical Nutrition 2018:nqy158-nqy. doi: 10.1093/ajcn/nqy158.
22. Wang Z M, Deurenberg P, Guo S S, Pietrobelli A, Wang J, Pierson J, R. N., Heymsfield S B. Six-compartment body composition model: Inter-method comparisons of total body fat measurement. International Journal of Obesity & Related Metabolic Disorders 1998; 22:329.
23. Cole K S. Permeability and Impermeability of Cell Membranes For Ions. Cold Spring Harbor Symposia on Quantitative Biology 1940; 8:110-22. doi: 10.1101/sqb.1940.008.01.013.
24. Hanai T. Electrical Properties of Emulsions, Emulsion science. London; New York: Academic Press, 1968.
25. Kyle U G, Bosaeus I, De Lorenzo A D, Deurenberg P, Elia M, Gómez J M, Heitmann B L, Kent-Smith L, Melchior J-C, Pirlich M, et al. Bioelectrical impedance analysis-part I: review of principles and methods. Clinical Nutrition 2004; 23:1226-43. doi: 10.1016/j.clnu.2004.06.004.
26. Wang Z M, Xavier P-S, Kotler D P, Wielopolski L, Withers R T, Pierson J, Heymsfield S B. Multicomponent methods: Evaluation of new and traditional soft tissue mineral models by in vivo neutron activation analysis. American Journal of Clinical Nutrition 2002; 76:968-74.
27. Jenkins N D, Miller J M, Buckner S L, Cochrane K C, Bergstrom H C, Hill E C, Smith C M, Housh T J, Cramer J T. Test-Retest Reliability of Single Transverse versus Panoramic Ultrasound Imaging for Muscle Size and Echo Intensity of the Biceps Brachii. Ultrasound Med Biol 2015; 41(6):1584-91. doi: 10.1016/j.ultrasmedbio.2015.01.017.
28. Bemben M G. Use of diagnostic ultrasound for assessing muscle size. Journal of Strength and Conditioning Research/National Strength & Conditioning Association 2002; 16:103-8.
29. Meylan C M, Nosaka K, Green J, Cronin J B. Temporal and kinetic analysis of unilateral jumping in the vertical, horizontal, and lateral directions. J Sports Sci 2010; 28(5):545-54. doi: 10.1080/02640411003628048.
30. Harry J R, Barker L A, James R, Dufek J S. Performance Differences Among Skilled Soccer Players of Different Playing Positions During Vertical Jumping and Landing. J Strength Cond Res 2018; 32(2):304-12. doi: 10.1519/jsc.0000000000002343.
31. Stock M S, Luera M J. Consistency of peak and mean concentric and eccentric force using a novel squat testing device. Journal of Applied Biomechanics 2014; 30:322-5. doi: 10.1123/jab.2013-0191.
32. Palmer T B, Pineda J G, Durham R M. Effects of Knee Position on the Reliability and Production of Maximal and Rapid Strength Characteristics During an Isometric Squat Test. J Appl Biomech 2017:1-23. doi: 10.1123/jab.2017-0213.
33. NSCA. Essentials of Strength Training and Conditioning. 4th ed. Champaign, III: Human Kinetics, 2016.
34. Compher C, Frankenfield D, Keim N, Roth-Yousey L Best Practice Methods to Apply to Measurement of Resting Metabolic Rate in Adults: A Systematic Review. J Am Diet Assoc 2006; 106:881-903. doi: DOI: 10.1016/j.jada.2006.02.009.
35. Martin S S, Blaha M J, Elshazly M B, et al. Comparison of a novel method vs the friedewald equation for estimating low-density lipoprotein cholesterol levels from the standard lipid profile. JAMA 2013; 310(19):2061-8. doi: 10.1001/jama.2013.280532.
36. Salimetrics L. Saliva Collection Handbook. 2015.
37. Stalder T, Kirschbaum C, Kudielka B M, Adam E K, Pruessner J C, Wust 5, Dockray S, Smyth N, Evans P, Hellhammer D H, et al. Assessment of the cortisol awakening response: Expert consensus guidelines. Psychoneuroendocrinology 2016; 63:414-32. doi: 10.1016/j.psyneuen.2015.10.010.
38. Angold A, Costello E J, Messer S C, Pickles A. Development of a short questionnaire for use in epidemiological studies of depression in children and adolescents. International Journal of Methods in Psychiatric Research 1995; 5(4):237-49.
39. Buysse D J, Reynolds C F, 3rd, Monk T H, Berman S R, Kupfer D J. The Pittsburgh Sleep Quality Index: a new instrument for psychiatric practice and research. Psychiatry Res 1989; 28(2):193-213.
40. Lauzon Bd, Romon M, Deschamps V, Lafay L, Borys J-M, Karlsson J, Ducimetiere P, Charles M A, Group FLVSFS. The Three-Factor Eating Questionnaire-R18 Is Able to Distinguish among Different Eating Patterns in a General Population. The Journal of Nutrition 2004; 134:2372-80.
41. Mattson M P, Longo V D, Harvie M. Impact of intermittent fasting on health and disease processes. Ageing Res Rev 2016. doi: 10.1016/j.arr.2016.10.005.
42. Rothschild J, Hoddy K K, Jambazian P, Varady K A. Time-restricted feeding and risk of metabolic disease: a review of human and animal studies. Nutrition reviews 2014. doi: 10.1111/nure.12104.
43. Sutton E F, Beyl R, Early K S, Cefalu W T, Ravussin E, Peterson C M. Early Time-Restricted Feeding Improves Insulin Sensitivity, Blood Pressure, and Oxidative Stress Even without Weight Loss in Men with Prediabetes. Cell Metabolism. doi: 10.1016/j.cmet.2018.04.010.
44. Stote K S, Baer D J, Spears K, Paul D R, Harris G K, Rumpler W V, Strycula P, Najjar S S, Ferrucci L, Ingram D K, et al. A controlled trial of reduced meal frequency without caloric restriction in healthy, normal-weight, middle-aged adults. The American Journal of Clinical Nutrition 2007; 85:981-8.
45. Gill S, Panda S. A Smartphone App Reveals Erratic Diurnal Eating Patterns in Humans that Can Be Modulated for Health Benefits. Cell Metabolism 2015; 22(5):789-98. doi: https://doi.org/10.1016/i.cmet.2015.09.005.
46. Wilkinson D J, Hossain T, Hill D S, Phillips B E, Crossland H, Williams J, Loughna P, Churchward-Venne T A, Breen L, Phillips S M, et al. Effects of leucine and its metabolite beta-hydroxy-beta-methylbutyrate on human skeletal muscle protein metabolism. J Physiol 2013; 591(11):2911-23. doi: 10.1113/jphysiol.2013.253203.
47. Wilson J M, Fitschen P J, Campbell B, Wilson G J, Zanchi N, Taylor L, Wilborn C, Kalman D S, Stout J R, Hoffman J R, et al. International Society of Sports Nutrition Position Stand: beta-hydroxy-beta-methylbutyrate (HMB). Journal of the International Society of Sports Nutrition 2013; 10:6. doi: 10.1186/1550-2783-10-6.
48. Hasselgren P O. beta-Hydroxy-beta-methylbutyrate (HMB) and prevention of muscle wasting. Metabolism 2014; 63(1):5-8. doi: 10.1016/j.metabol.2013.09.015.
49. Sanchez-Martinez J, Santos-Lozano A, Garcia-Hermoso A, Sadarangani K P, Cristi-Montero C. Effects of beta-hydroxy-beta-methylbutyrate supplementation on strength and body composition in trained and competitive athletes: A meta-analysis of randomized controlled trials. Journal of Science and Medicine in Sport 2018; 21(7):727-35. doi: https://doi.org/10.1016/j.jsams.2017.11.003.
50. Livingstone M B, Prentice A M, Strain J J, Coward W A, Black A E, Barker M E, McKenna P G, Whitehead R G. Accuracy of weighed dietary records in studies of diet and health. BMJ (Clinical research ed) 1990; 300(6726):708-12.
51. Sawaya A L, Tucker K, Tsay R, Willett W, Saltzman E, Dallal G E, Roberts S B. Evaluation of four methods for determining energy intake in young and older women: comparison with doubly labeled water measurements of total energy expenditure. Am J Clin Nutr 1996; 63(4):491-9. doi: 10.1093/ajcn/63.4.491.
52. Ng, B. K. et al. Validation of rapid 4-component body composition assessment with the use of dual-energy X-ray absorptiometry and bioelectrical impedance analysis. Am J Clin Nutr 108, 708-715, doi:10.1093/ajcn/nqy158 (2018).
53. Wilson, J. P., Strauss, B. J., Fan, B., Duewer, F. W. & Shepherd, J. A. Improved 4-compartment body-composition model for a clinically accessible measure of total body protein. Am J Clin Nutr 97, 497-504, doi:10.3945/ajcn.112.048074 (2013).
54. Panton, L. B., Rathmacher, J. A., Baier, S. & Nissen, S. Nutritional supplementation of the leucine metabolite β-hydroxy β-methylbutyrate (HMB) during resistance training. Nutrition 16, 734-739 (2000).
55. Pollock, M. L. W., J. H., Exercise in Health and Disease: Evaluation and Prescription for Prevention and Rehabilitation., 319 (W B Saunders Co, 1990).
56. Siri, W. E. in Technique for measuring Body Composition (eds Brozek. J. & A. Henschel) 223-224 (National Academy of Science, 1961).

Supplemental Table 1.
Participant Compliance.

| | PP | | | | ITT | | | |
|---|---|---|---|---|---|---|---|---|
| | CD | TRF_{HMB} | TRF | P (group) | CD | TRF_{HMB} | TRF | P (group) |
| Meal Timing Compliance (%) | 98 ± 5 | 93 ± 6 | 91 ± 3 | 0.03* | 98 ± 5 | 92 ± 7 | 89 ± 8 | 0.47 |
| Protein Supplementation (scoops/d) | 1.8 ± 0.3 | 1.6 ± 0.3 | 1.4 ± 0.4 | 0.08 | 1.8 ± 0.2 | 1.6 ± 0.3 | 1.5 ± 0.4 | 0.53 |
| Capsule Supplementation (servings/d) | 2.7 ± 0.2 | 2.8 ± 0.1 | 2.7 ± 0.1 | 0.28 | 2.7 ± 0.2 | 2.8 ± 0.2 | 2.6 ± 0.2 | 0.09 |
| Compliance: Capsule count (%) | 87 ± 11 | 87 ± 6 | 88 ± 8 | 0.95 | 86 ± 11 | 84 ± 13 | 85 ± 10 | 0.29 |
| Compliauce: Capsules self-report (%) | 90 ± 6 | 94 ± 5 | 89 ± 6 | 0.22 | 91 ± 7 | 90 ± 7 | 87 ± 8 | 0.09 |

Mean ± SD; P values from one-way ANOVA.
*Meal timing compliance was higher in CD than TRF (p = 0.03), but did not differ between CD and TRF_{HMB} (p = 0.29) or between TRF_{HMB} and TRF (p = 0.96).
CD: control diet; ITT; intention-to-treat; PP: per protocol; TRF: time-restricted feeding; TRF_{HMB}: time-restricted feeding plus beta-hydroxy beta-methylbutyrate supplementation.

Supplemental Table 2.
Urinary HMB concentrations.

| | | Pre- | | P | P | P |
|---|---|---|---|---|---|---|
| Analysis | Group | intervention^{a} | Intervention^{a} | (group) | (time) | (I) |
| ITT | CD | 102 ± 20 | 135 ± 721 | <0.0001* | 0.001* | <0.0001* |
| | TRF_{HMB} | 108 ± 21 | 5033 ± 751 | | | |
| | TRF | 134 ± 21 | 60 ± 751 | | | |
| PP | CD | 89 ± 28 | 116 ± 985 | <0.001* | 0.002* | <0.001* |
| | TRF_{HMB} | 106 ± 32 | 6553 ± 1117 | | | |
| | TRF | 163 ± 30 | 10 ± 1045 | | | |

*Statistically significant (p < 0.05);
^{a}Values shown in nmol/mL.
CD: control diet; I: interaction; ITT: intention-to-treat; PP: per protocol; TRF: time-restricted feeding; TRF_{HMB}: time-restricted feeding plus beta-hydroxy beta-methylbutyrate supplementation

Supplemental Table 3.
Timing of Eating Windows.

| | PP | | | | | | | ITT |
|---|---|---|---|---|---|---|---|---|
| | | Pre- | | | P | P | P | Pre- |
| | Group | Intervention | Intervention | Δ | (group) | (time) | (I) | intervention |
| First Eating | CD | 8:11 ± 1:54 | 8:45 ± 0:53 | 0:34 | <0.0001*^{a} | 0.002*^{b} | 0.09 | 8:43 ± 1:54 |
| Occasion^{a} | TRF_{HMB} | 11:15 ± 1:14 | 12:13 ± 0:17 | 0:58 | | | | 9:40 ± 2:05 |
| | TRF | 9:33 ± 2:35 | 12:09 ± 0:25 | 2:36 | | | | 10:16 ± 2:14 |
| Last Eating | CD | 20:13 ± 1:03 | 22:02 ± 1:17 | 1:49 | 0.005* | 0.78 | 0.001*^{c} | 20:04 ± 1:14 |
| Occasion^{a} | TRF_{HMB} | 21:06 ± 1:50 | 19:37 ± 0:37 | −1:29 | | | | 20:41 ± 1:34 |
| | TRF | 19:45 ± 0:53 | 19:40 ± 0:20 | −0:05 | | | | 19:43 ± 1:03 |
| Eating Window | CD | 12.0 ± 2.1 | 13.3 ± 1.8 | 1.3 | <0.0001* | 0.02* | 0.006*^{d} | 11.3 ± 2.5 |
| (h) | TRF_{HMB} | 9.9 ± 2.5 | 7.3 ± 0.6 | −2.6 | | | | 11.0 ± 2.5 |
| | TRF | 10.2 ± 3.1 | 7.5 ± 0.5 | −2.7 | | | | 9.5 ± 2.7 |
| Eating frequency | CD | 4.7 ± 1.1 | 5.2 ± 1.4 | 0.5 | 0.35 | 0.16 | 0.38 | 4.2 ± 1.5 |
| (times/d) | TRF_{HMB} | 3.8 ± 0.6 | 4.6 ± 1.1 | 0.8 | | | | 3.9 ± 1.4 |
| | TRF | 4.6 ± 1.0 | 4.4 ± 0.9 | −0.2 | | | | 4.1 ± 1.2 |

| | | ITT | | | | |
|---|---|---|---|---|---|---|
| | PP | | | P | P | P |
| | Group | Intervention | Δ | (group) | (time) | (I) |
| First Eating | CD | 8:46 ± 0:58 | 0:03 | <0.0001* | <0.0001* | 0.009*^{e} |
| Occasion^{a} | TRF_{HMB} | 12:06 ± 0:22 | 2:26 | | | |
| | TRF | 12:06 ± 0:25 | 1:50 | | | |
| Last Eating | CD | 21:59 ± 1:06 | 1:55 | <0.001* | 0.28 | <0.0001*^{c} |
| Occasion^{a} | TRF_{HMB} | 19:41 ± 0:30 | −1:00 | | | |
| | TRF | 19.36 ± 0:25 | −0:07 | | | |
| Eating Window | CD | 13.2 ± 1.6 | 1.9 | <0.0001* | 0.005 | <0.0001*^{d} |
| (h) | TRF_{HMB} | 7.6 ± 0.7 | −3.4 | | | |
| | TRF | 7.5 ± 0 6 | −2.0 | | | |
| Eating frequency | CD | 5.1 ± 1.3 | 0.9 | 0.53 | 0.01*^{d} | 0.41 |
| (times/d) | TRF_{HMB} | 4.6 ± 0.9 | 0.7 | | | |
| | TRF | 4.3 ± 1.0 | 0.2 | | | |

Mean ± SD; P values from mixed model analysis.
^{a}Eating occasions shown in hh:mm.
^{*}Statistically significant (p < 0.05);
^{a}Significantly different than CD in both TRF and TRF_{HMB};
^{b}Significantly different between pre-intervention and intervention;
^{c}During the intervention, the timing of the last eating occasion was later in CD than both TRF and TRF_{HMB},
^{d}As compared to pre-intervention the intervention eating window was longer in CD but shorter in TRF and TRF_{HMB};
^{e}The timing of the first eating occasion was later during the intervention than the pre-intervention period for TRF and TRF_{HMB}, but did not differ in CD.
CD: control diet; I: interaction; ITT: intention-to-treat; PP: per protocol; TRF: time-restricted feeding; TRF_{HMB}: time-restricted feeding plus beta-hydroxy beta-methylbutyrate supplementation

Supplemental Table 4.
Workout Volume.

| | PP | | | | | | | | |
|---|---|---|---|---|---|---|---|---|---|
| | | | | | Δ | ES | P | P | P |
| | Group | First 4 weeks | Second 4 weeks | Δ | (%) | (d) | (group) | (time) | (I) |
| UB Session | CD | 2670 ± 267 | 3347 ± 262 | 677 | 25 | 0.85 | 0.20 | <0.0001* | 0.92 |
| Volume (kg) | TRF_{HMB} | 2869 ± 303 | 3463 ± 297 | 594 | 21 | 0.75 | | | |
| | TRF | 3369 ± 283 | 3996 ± 278 | 627 | 19 | 0.79 | | | |
| LS Session | CD | 8249 ± 833 | 10438 ± 1130 | 2189 | 27 | 0.74 | 0.65 | <0.0001* | 0.47 |
| Volume (kg) | TRF_{HMB} | 9434 ± 944 | 11915 ± 1281 | 2481 | 26 | 0.83 | | | |
| | TRF | 9434 ± 883 | 10857 ± 1198 | 1423 | 15 | 0.48 | | | |
| UB Volume (kg) | CD | 15240 ± 1633 | 17752 ± 1542 | 2512 | 16 | 0.53 | 0.12 | 0.08 | 0.78 |
| | TRF_{HMB} | 16696 ± 1851 | 17923 ± 1749 | 1227 | 7 | 0.26 | | | |
| | TRF | 20217 ± 1732 | 21384 ± 1636 | 1167 | 6 | 0.24 | | | |
| LB Volume (kg) | CD | 47196 ± 5048 | 54970 ± 5523 | 7774 | 16 | 0.47 | 0.58 | 0.08 | 0.65 |
| | TRF_{HMB} | 54729 ± 5724 | 60345 ± 6263 | 5616 | 10 | 0.34 | | | |
| | TRF | 56603 ± 5354 | 58340 ± 5858 | 1737 | 3 | 0.10 | | | |
| | | ITT | | | | | | | |
| | PP | | | | Δ | ES | P | P | P |
| | Group | First 4 weeks | Second 4 weeks | Δ | (%) | (d) | (group) | (time) | (I) |
| UB Session | CD | 2633 ± 202 | 3315 ± 212 | 682 | 26 | 0.88 | 0.38 | <0.0001* | 0.92 |
| Volume (kg) | TRF_{HMB} | 2722 ± 210 | 3336 ± 215 | 614 | 23 | 0.80 | | | |
| | TRF | 3028 ± 215 | 3668 ± 210 | 640 | 21 | 0.84 | | | |
| LS Session | CD | 8166 ± 633 | 10340 ± 890 | 2174 | 27 | 0.75 | 0.80 | <0.0001* | 0.38 |
| Volume (kg) | TRF_{HMB} | 8734 ± 659 | 10978 ± 902 | 2244 | 26 | 0.79 | | | |
| | TRF | 8508 ± 659 | 9921 ± 882 | 1413 | 17 | 0.50 | | | |
| UB Volume (kg) | CD | 15353 ± 1265 | 17816 ± 1329 | 2463 | 16 | 0.51 | 0.29 | 0.005* | 0.91 |
| | TRF_{HMB} | 15715 ± 1317 | 17437 ± 1298 | 1722 | 11 | 0.37 | | | |
| | TRF | 17777 ± 1317 | 19879 ± 1223 | 2102 | 12 | 0.46 | | | |
| LB Volume (kg) | CD | 47727 ± 3946 | 55346 ± 4742 | 7619 | 16 | 0.47 | 0.92 | 0.01* | 0.78 |
| | TRF_{HMB} | 50374 ± 4107 | 56873 ± 4655 | 6499 | 13 | 0.41 | | | |
| | TRF | 49936 ± 4107 | 53963 ± 4412 | 4027 | 8 | 0.26 | | | |

Mean ± SE; P values from mixed model analysis
*Statistically significant (p < 0.05) difference between first and second 4 weeks of intervention
CD: control diet; ES: effect size; I: interaction; ITT: intention-to-treat; LB; lower body; PP; per protocol; TRF: time-restricted feeding; TRF_{HMB}: time-restricted feeding plus beta-hydroxy beta-methylbutyrate supplementation; UB: upper body

Supplemental Table 5.
Physical Activity.

| | PP | | | | | | | | | |
|---|---|---|---|---|---|---|---|---|---|---|
| | | Pre- | Early | Late | | Δ | ES | P | P | P |
| | Group | intervention | intervention | Intervention | Δ | (%) | (d) | (group) | (time) | (I) |
| PAEE | CD | 301 ± 64 | 329 ± 55 | 353 ± 70 | 52 | 17 | 0.26 | 0.50 | 0.54 | 0.09 |
| (kcal) | TRF_{HMB} | 438 ± 73 | 453 ± 61 | 409 ± 80 | −29 | −7 | 0.28 | | | |
| | TRF | 423 ± 67 | 340 ± 59 | 307 ± 75 | −116 | −27 | −0.73 | | | |
| Sedentary | CD | 583 ± 18 | 551 ± 22 | 530 ± 28 | −53 | −9 | −0.75 | 0.87 | 0.87 | 0.02*^{a} |
| Time | TRF_{HMB} | 556 ± 20 | 546 ± 23 | 561 ± 31 | 5 | 1 | 0.07 | | | |
| (min/d) | TRF | 516 ± 18 | 558 ± 23 | 547 ± 29 | 31 | 6 | 0.45 | | | |
| LI PA | CD | 201 ± 15 | 239 ± 20 | 244 ± 26 | 43 | 25 | 0.68 | 0.58 | 0.90 | 0.04*^{a} |
| (min/d) | TRF_{HMB} | 214 ± 17 | 222 ± 22 | 208 ± 29 | −6 | −3 | −0.10 | | | |
| | TRF | 264 ± 16 | 230 ± 21 | 239 ± 27 | −25 | −9 | −0.40 | | | |
| MV PA | CD | 41 ± 10 | 36 ± 9 | 42 ± 11 | 1 | 2 | 0.03 | 0.50 | 0.32 | 0.17 |
| (min/d) | TRF_{HMB} | 52 ± 11 | 58 ± 10 | 48 ± 13 | −4 | −8 | −0.13 | | | |
| | TRF | 45 ± 10 | 35 ± 10 | 27 ± 12 | −18 | −40 | −0.58 | | | |
| Steps | CD | 7075 ± 1222 | 7689 ± 924 | 8182 ± 1377 | 1107 | 16 | 0.28 | 0.68 | 0.65 | 0.19 |
| (#/d) | TRF_{HMB} | 9286 ± 1379 | 9385 ± 984 | 8274 ± 1596 | −1012 | −11 | −0.26 | | | |
| | TRF | 9045 ± 1256 | 7011 ± 977 | 7696 ± 1474 | −1349 | −15 | −0.35 | | | |

| | | ITT | | | | | | | | |
|---|---|---|---|---|---|---|---|---|---|---|
| | PP | Pre- | Early | Late | | Δ | ES | P | P | P |
| | Group | intervention | Intervention | Intervention | Δ | (%) | (d) | (group) | (time) | (I) |
| PAEE | CD | 325 ± 49 | 326 ± 41 | 359 ± 54 | 34 | 10 | 0.18 | 0.77 | 0.43 | 0.034*^{b} |
| (kcal) | TRF_{HMB} | 378 ± 49 | 411 ± 41 | 352 ± 52 | −26 | −7 | −0.14 | | | |
| | TRF | 403 ± 48 | 325 ± 43 | 315 ± 52 | −88 | −22 | −0.49 | | | |
| Sedentary | CD | 568 ± 17 | 561 ± 19 | 543 ± 24 | −25 | −4 | −0.32 | 0.76 | 0.27 | 0.048*^{c} |
| Time | TRF_{HMB} | 574 ± 16 | 570 ± 19 | 570 ± 23 | −4 | −1 | −0.06 | | | |
| (min/d) | TRF | 523 ± 16 | 568 ± 20 | 572 ± 23 | 49 | 9 | 0.69 | | | |
| LI PA | CD | 217 ± 16 | 224 ± 18 | 243 ± 25 | 26 | 12 | 0.33 | 0.55 | 0.33 | 0.048*^{c} |
| (min/d) | TRF_{HMB} | 203 ± 16 | 200 ± 18 | 210 ± 24 | 7 | 3 | 0.10 | | | |
| | TRF | 256 ± 16 | 221 ± 19 | 198 ± 23 | −58 | −23 | −0.81 | | | |
| MV PA | CD | 38 ± 7 | 34 ± 7 | 39 ± 9 | 1 | 3 | 0.03 | 0.42 | 0.60 | 0.075 |
| (min/d) | TRF_{HMB} | 48 ± 7 | 57 ± 7 | 44 ± 9 | −4 | −8 | −0.14 | | | |
| | TRF | 46 ± 7 | 37 ± 7 | 38 ± 9 | −8 | −17 | −0.28 | | | |
| Steps | CD | 7372 ± 859 | 7166 ± 810 | 8132 ± 1143 | 760 | 10 | 0.20 | 0.71 | 0.50 | 0.06 |
| (#/d) | TRF_{HMB} | 8393 ± 855 | 9179 ± 790 | 7896 ± 1094 | −497 | −6 | −0.14 | | | |
| | TRF | 9217 ± 841 | 7310 ± 850 | 7942 ± 1062 | −1275 | −14 | −0.37 | | | |

Mean ± SE; P values from mixed model analysis
*Statistically significant (p < 0.05);
^{a}Value differed between CD than TRF during the pre-intervention period, with no differences between groups during the intervention;
^{b}Simple main effect for time in TRF only, but comparisons between time points were not statistically significant;
^{c}Value differed between early intervention than pre-intervention in TRF only;
^{d}Pre-intervention LI PA was higher in TRF than TRF_{HMB} and LI PA decreased from pre-intervcntion to early intervention in TRF only
CD; control diet; ES: effect size; I: interaction; ITT; intention-to-treat; LI PA: light-intensity physical activity; MVPA: moderate- or vigorous-intensity physical activity; PAEE; physical activity energy expenditure; PP: per protocol; TRF: time-restricted feeding; TRF_{HMB}: time-restricted feeding plus beta-hydroxy beta-methylbutyrate supplementation

Supplemental Table 6.
Rate of Force Development.
PP

| Knee | | | | | | | Δ | ES | P | P | P |
|---|---|---|---|---|---|---|---|---|---|---|---|
| Angle | Variable | Intervention | Baseline | W 4 | W 8 | Δ | (%) | (d) | (group) | (time) | (I) |
| 120° | RFD_{30 ms} | CD | 2563 ± 584 | 2178 ± 638 | 2947 ± 595 | 384 | 15 | 0.22 | 0.62 | 0.04*^{a} | 0.80 |
| | | TRF_{HMB} | 1669 ± 662 | 2267 ± 723 | 2917 ± 675 | 1248 | 75 | 0.71 | | | |
| | | TRF | 2618 ± 619 | 3017 ± 676 | 3556 ± 649 | 938 | 36 | 0.52 | | | |
| | RFD_{50 ms} | CD | 3078 ± 668 | 2479 ± 694 | 3340 ± 596 | 262 | 9 | 0.14 | 0.66 | 0.03*^{a} | 0.65 |
| | | TRF_{HMB} | 2016 ± 758 | 3028 ± 787 | 3470 ± 675 | 1454 | 72 | 0.77 | | | |
| | | TRF | 3079 ± 709 | 3516 ± 736 | 4074 ± 647 | 995 | 32 | 0.52 | | | |
| | RFD_{100 ms} | CD | 3909 ± 1017 | 3382 ± 1054 | 3868 ± 980 | −41 | −1 | −0.01 | 0.54 | 0.20 | 0.74 |
| | | TRF_{HMB} | 2642 ± 1153 | 3845 ± 1195 | 4629 ± 1111 | 1987 | 75 | 0.66 | | | |
| | | TRF | 4635 ± 1078 | 5053 ± 1117 | 5338 ± 1057 | 703 | 15 | 0.23 | | | |
| | RFD_{200 ms} | CD | 3668 ± 606 | 3630 ± 571 | 3836 ± 502 | 168 | 5 | 0.10 | 0.32 | 0.57 | 0.53 |
| | | TRF_{HMB} | 4079 ± 688 | 4636 ± 648 | 4824 ± 569 | 745 | 18 | 0.45 | | | |
| | | TRF | 5127 ± 643 | 4437 ± 606 | 4780 ± 548 | −347 | −7 | −0.21 | | | |
| 150° | RFD_{30 ms} | CD | 2618 ± 679 | 2418 ± 552 | 3192 ± 549 | 574 | 22 | 0.31 | 0.78 | 0.19 | 0.32 |
| | | TRF_{HMB} | 2121 ± 770 | 2845 ± 625 | 3177 ± 622 | 1056 | 50 | 0.57 | | | |
| | | TRF | 2705 ± 720 | 3491 ± 585 | 3407 ± 596 | 702 | 26 | 0.38 | | | |
| | RFD_{50 ms} | CD | 3416 ± 1047 | 2764 ± 630 | 3515 ± 532 | 99 | 3 | 0.04 | 0.93 | 0.48 | 0.13 |
| | | TRF_{HMB} | 2420 ± 1187 | 3274 ± 715 | 3572 ± 604 | 1152 | 48 | 0.46 | | | |
| | | TRF | 2963 ± 1110 | 3925 ± 669 | 3515 ± 575 | 552 | 19 | 0.22 | | | |
| | RFD_{100 ms} | CD | 3690 ± 1215 | 3664 ± 1039 | 4798 ± 943 | 1108 | 30 | 0.34 | 0.69 | 0.24 | 0.61 |
| | | TRF_{HMB} | 3072 ± 1377 | 3940 ± 1178 | 4346 ± 1070 | 1274 | 41 | 0.39 | | | |
| | | TRF | 4346 ± 1288 | 5348 ± 1102 | 5268 ± 1022 | 922 | 21 | 0.28 | | | |
| | RFD_{200 ms} | CD | 3106 ± 625 | 3417 ± 559 | 4196 ± 492 | 1090 | 35 | 0.65 | 0.36 | 0.06 | 0.53 |
| | | TRF_{HMB} | 3441 ± 708 | 3613 ± 634 | 4237 ± 558 | 796 | 23 | 0.47 | | | |
| | | TRF | 4785 ± 663 | 4394 ± 593 | 4520 ± 534 | −265 | −6 | −0.16 | | | |

| PP | | | ITT | | | | | | | | |
|---|---|---|---|---|---|---|---|---|---|---|---|
| Knee | | | | | | | Δ | ES | P | P | P |
| Angle | Variable | Intervention | Baseline | W 4 | W 8 | Δ | (%) | (d) | (group) | (time) | (I) |
| 120° | RFD_{30 ms} | CD | 2248 ± 396 | 1987 ± 430 | 2794 ± 496 | 546 | 24 | 0.33 | 0.79 | 0.02*^{b} | 0.58 |
| | | TRF_{HMB} | 1638 ± 411 | 2131 ± 457 | 2606 ± 494 | 969 | 59 | 0.59 | | | |
| | | TRF | 2067 ± 411 | 2555 ± 457 | 2859 ± 482 | 792 | 38 | 0.49 | | | |
| | RFD_{50 ms} | CD | 2602 ± 455 | 2215 ± 473 | 3106 ± 507 | 504 | 19 | 0.28 | 0.87 | 0.02*^{b} | 0.44 |
| | | TRF_{HMB} | 2017 ± 472 | 2760 ± 506 | 3153 ± 510 | 1135 | 56 | 0.64 | | | |
| | | TRF | 2482 ± 472 | 2988 ± 506 | 3250 ± 499 | 769 | 31 | 0.44 | | | |
| | RFD_{100 ms} | CD | 3206 ± 693 | 2858 ± 713 | 3413 ± 756 | 206 | 6 | 0.08 | 0.69 | 0.14 | 0.49 |
| | | TRF_{HMB} | 2621 ± 719 | 3451 ± 759 | 4205 ± 774 | 1585 | 60 | 0.59 | | | |
| | | TRF | 3598 ± 719 | 4103 ± 759 | 4104 ± 763 | 506 | 14 | 0.19 | | | |
| | RFD_{200 ms} | CD | 3266 ± 484 | 3182 ± 449 | 3496 ± 471 | 230 | 7 | 0.13 | 0.37 | 0.70 | 0.51 |
| | | TRF_{HMB} | 3661 ± 503 | 4121 ± 475 | 4332 ± 472 | 671 | 18 | 0.38 | | | |
| | | TRF | 4249 ± 503 | 4021 ± 475 | 3925 ± 463 | −324 | −8 | −0.19 | | | |
| 150° | RFD_{30 ms} | CD | 2167 ± 454 | 2141 ± 394 | 2980 ± 468 | 814 | 38 | 0.46 | 0.85 | 0.02*^{b} | 0.30 |
| | | TRF_{HMB} | 1922 ± 471 | 2612 ± 420 | 2851 ± 469 | 929 | 48 | 0.53 | | | |
| | | TRF | 2159 ± 471 | 2961 ± 420 | 3008 ± 459 | 849 | 39 | 0.49 | | | |
| | RFD_{50 ms} | CD | 2729 ± 677 | 2442 ± 451 | 3285 ± 466 | 556 | 20 | 0.25 | 0.95 | 0.11 | 0.19 |
| | | TRF_{HMB} | 2203 ± 703 | 2989 ± 482 | 3208 ± 468 | 1004 | 46 | 0.45 | | | |
| | | TRF | 2352 ± 703 | 3395 ± 482 | 3215 ± 458 | 862 | 37 | 0.39 | | | |
| | RFD_{100 ms} | CD | 2971 ± 795 | 3035 ± 707 | 4311 ± 746 | 1341 | 45 | 0.45 | 0.69 | 0.03*^{b} | 0.27 |
| | | TRF_{HMB} | 2694 ± 825 | 3567 ± 750 | 3944 ± 754 | 1251 | 46 | 0.42 | | | |
| | | TRF | 3295 ± 825 | 4613 ± 750 | 4522 ± 741 | 1227 | 37 | 0.42 | | | |
| | RFD_{200 ms} | CD | 2693 ± 473 | 2979 ± 418 | 3863 ± 442 | 1170 | 43 | 0.66 | 0.31 | 0.03*^{b} | 0.60 |
| | | TRF_{HMB} | 3163 ± 491 | 3511 ± 447 | 3876 ± 443 | 713 | 23 | 0.41 | | | |
| | | TRF | 3717 ± 491 | 4102 ± 447 | 4216 ± 433 | 499 | 13 | 0.29 | | | |

Mean ± SE; P values from mixed model analysis
*Statistically significant (p < 0.05);
^{a}Pairwise comparisons between time points were not statistically significant;
^{b}Value at W 8 was higher than baseline in all groups combined
CD: control diet; ES: effect size; I: interaction; ITT: intention-to-treat; PP: per protocol; RFD: rate of force development; TRF: time-restricted feeding; TRF_{HMB}: time-restricted feeding plus beta-hydroxy beta-methylbutyrate supplementation. Time subscript on RFD represents duration over which RFD was calculated; W 4: week 4; W 8; week 8.

Supplemental Table 7.
Vertical Jump Performance

| | PP | | | | | | | | |
|---|---|---|---|---|---|---|---|---|---|
| | | | | | Δ | ES | P | P | P |
| | Group | Baseline | W 8 | Δ | (%) | (d) | (group) | (time) | (I) |
| Jump | CD | 0.28 ± 0.03 | 0.33 ± 0.03 | 0.05 | 18 | 0.63 | 0.09 | 0.43 | 0.48 |
| Height | TRF_{HMB} | 0 22 ± 0.03 | 0.24 ± 0 03 | 0.02 | 9 | 0.21 | | | |
| | TRF | 0.28 ± 0.03 | 0.26 ± 0.03 | −0.02 | −7 | −0.16 | | | |
| | | ITT | | | | | | | |
| | PP | | | | Δ | ES | P | P | P |
| | Group | Baseline | W 8 | Δ | (%) | (d) | (group) | (time) | (I) |
| Jump | CD | 0.27 ± 0.02 | 0.33 ± 0.03 | 0.06 | 22 | 0.63 | 0.09 | 0.06 | 0.45 |
| Height | TRF_{HMB} | 0.21 ± 0.02 | 0.27 ± 0.03 | 0.06 | 29 | 0.65 | | | |
| | TRF | 0.25 ± 0 02 | 0 25 ± 0.03 | 0.00 | 0 | 0.00 | | | |

Mean ± SE; P values from mixed model analysis
*Statistically significant difference between baseline and W 8 values in all groups combined
CD: conlrol diet; ES: effect size; I: interaction; ITT: intention-to-treat; PP: per protocol; TRF: time-restricted feeding; TRF_{HMB}: time-restricted feeding plus beta-hydroxy beta-methylbutyrate supplementation; W 8: week 8.

Supplemental Table 8.
Metabolism

| | PP | | | | | | | | |
|---|---|---|---|---|---|---|---|---|---|
| | | | | | Δ | ES | P | P | P |
| | Group | Baseline | W 8 | Δ | (%) | (d) | (group) | (time) | (I) |
| RMR | CD | 1486 ± 54 | 1441 ± 50 | −45 | −3 | −0.29 | 0.43 | 0.44 | 0.23 |
| (kcal/d) | TRF_{HMB} | 1472 ± 61 | 1519 ± 57 | 47 | 3 | 0.30 | | | |
| | TRF | 1586 ± 57 | 1523 ± 53 | −63 | −4 | −0.40 | | | |
| RQ | CD | 0.89 ± 0.03 | 0.84 ± 0.02 | −0.05 | −6 | −0.65 | 0.02*^{a} | 0.13 | 0.24 |
| (au) | TRF_{HMB} | 0.81 ± 0.03 | 0.78 ± 0.02 | −0.03 | −4 | −0.44 | | | |
| | TRF | 0.80 ± 0.03 | 0.81 ± 0.02 | 0.01 | 1 | 0.14 | | | |
| | | ITT | | | | | | | |
| | PP | | | | Δ | ES | P | P | P |
| | Group | Baseline | W 8 | Δ | (%) | (d) | (group) | (time) | (I) |
| RMR | CD | 1548 ± 46 | 1477 ± 45 | −71 | −5 | −0.42 | 0.69 | 0.11 | 0.27 |
| (kcal/d) | TRF_{HMB} | 1466 ± 48 | 1481 ± 44 | 15 | 1 | 0.09 | | | |
| | TRF | 1549 ± 48 | 1495 ± 42 | −54 | −3 | −0.33 | | | |
| RQ | CD | 0.88 ± 0.02 | 0.83 ± 0.02 | −0.05 | −6 | −0.67 | 0.10 | 0.15 | 0.21 |
| (au) | TRF_{HMB} | 0.82 ± 0.02 | 0 79 ± 0.02 | −0.03 | −4 | −0.42 | | | |
| | TRF | 0.81 ± 0.02 | 0.83 ± 0.02 | 0.02 | 2 | 0.28 | | | |

Mean ± SE; P values from mixed model analysis
*Statistically significant difference between CD and TRF_{HMB} across both time points combined
CD: control diet; ES: effect size; I: interaction; ITT: intention-to-treat; PP: per protocol; RMR: resting metabolic rate; RQ: respiratory quotient; TRF: time-restricted feeding; TRF_{HMB}: time-restricted feeding plus beta-hydroxy beta-methylbutyrate supplementation; W 8: week 8.

Supplemental Table 9.
Blood Variables.

| | PP | | | | | | | | |
|---|---|---|---|---|---|---|---|---|---|
| | | | | | Δ | ES | P | P | P |
| | Group | Baseline | W 8 | Δ | (%) | (d) | (group) | (time) | (I) |
| Glucose | CD | 97 ± 4 | 91 ± 4 | −6 | −6 | −0.50 | 0.49 | 0.37 | 0.78 |
| (mg/dL) | TRF_{HMB} | 92 ± 4 | 90 ± 4 | −2 | −2 | −0.19 | | | |
| | TRF | 89 ± 4 | 89 ± 4 | 0 | 0 | 0.00 | | | |
| Cholesterol | CD | 177 ± 12 | 183 ± 14 | 6 | 3 | 0.15 | 0.84 | 0.10 | 0.38 |
| (mg/dL) | TRF_{HMB} | 168 ± 13 | 183 ± 15 | 15 | 9 | 0.40 | | | |
| | TRF | 168 ± 13 | 169 ± 15 | 1 | 1 | 0.03 | | | |
| HDL | CD | 73 ± 5 | 72 ± 6 | −1 | −1 | −0.06 | 0.37 | 0.92 | 0.67 |
| (mg/dL) | TRF_{HMB} | 72 ± 5 | 70 ± 6 | −2 | −3 | −0.14 | | | |
| | TRF | 61 ± 5 | 63 ± 6 | 2 | 3 | 0.13 | | | |
| Triglycerides | CD | 83 ± 12 | 76 ± 16 | −7 | −8 | −0.16 | 0.95 | 0.56 | 0.33 |
| (mg/dL) | TRF_{HMB} | 91 ± 14 | 80 ± 17 | −11 | −12 | −0.27 | | | |
| | TRF | 75 ± 14 | 84 ± 17 | 9 | 12 | 0.20 | | | |
| VLDL | CD | 17 ± 2 | 15 ± 3 | −2 | −12 | −0.26 | 0.95 | 0.51 | 0.30 |
| (mg/dL) | TRF_{HMB} | 18 ± 3 | 16 ± 3 | −2 | −11 | −0.30 | | | |
| | TRF | 15 ± 3 | 17 ± 3 | 2 | 13 | 0.24 | | | |
| Insulin | CD | 13 ± 2 | 13 ± 2 | 0 | 0 | 0.00 | 0.30 | 0.94 | 0.97 |
| (mcU/mL) | TRF_{HMB} | 9 ± 3 | 10 ± 3 | 1 | 11 | 0.55 | | | |
| | TRF | 9 ± 3 | 9 ± 3 | 0 | 0 | 0.34 | | | |
| LDL | CD | 86 ± 8 | 94 ± 9 | 8 | 9 | 0.31 | 0.96 | 0.04* | 0.09 |
| (mg/dL) | TRF_{HMB} | 78 ± 9 | 96 ± 9 | 18 | 23 | 0.76 | | | |
| | TRF | 91 ± 9 | 88 ± 9 | −3 | −3 | −0.12 | | | |
| | | ITT | | | | | | | |
| | PP | | | | Δ | ES | P | P | P |
| | Group | Baseline | W 8 | Δ | (%) | (d) | (group) | (time) | (I) |
| Glucose | CD | 93 ± 3 | 91 ± 4 | −2 | −2 | −0.15 | 0.69 | 0.69 | 0.92 |
| (mg/dL) | TRF_{HMB} | 90 ± 3 | 89 ± 3 | −1 | −1 | −0.09 | | | |
| | TRF | 89 ± 3 | 89 ± 3 | 0 | 0 | 0.00 | | | |
| Cholesterol | CD | 179 ± 9 | 185 ± 11 | 6 | 3 | 0.16 | 0.89 | 0.72 | 0.38 |
| (mg/dL) | TRF_{HMB} | 178 ± 9 | 183 ± 11 | 5 | 3 | 0.14 | | | |
| | TRF | 179 ± 10 | 172 ± 11 | −7 | −4 | −0.18 | | | |
| HDL | CD | 69 ± 4 | 69 ± 6 | 0 | 0 | 0.00 | 0.50 | 0.40 | 0.40 |
| (mg/dL) | TRF_{HMB} | 75 ± 4 | 68 ± 5 | −7 | −9 | −0.43 | | | |
| | TRF | 64 ± 4 | 65 ± 5 | 1 | 2 | 0.06 | | | |
| Triglycerides | CD | 83 ± 9 | 75 ± 13 | −8 | −10 | −0.19 | 0.67 | 0.40 | 0.32 |
| (mg/dL) | TRF_{HMB} | 95 ± 10 | 85 ± 12 | −10 | −11 | −0.25 | | | |
| | TRF | 88 ± 10 | 93 ± 12 | 5 | 6 | 0.13 | | | |
| VLDL | CD | 17 ± 2 | 15 ± 3 | −2 | −12 | −0.21 | 0.69 | 0.35 | 0.29 |
| (mg/dL) | TRF_{HMB} | 19 ± 2 | 17 ± 2 | −2 | −11 | −0.28 | | | |
| | TRF | 18 ± 2 | 19 ± 2 | 1 | 6 | 0.14 | | | |
| Insulin | CD | 12 ± 2 | 13 ± 4 | 1 | 8 | 0.03 | 0.85 | 0.43 | 0.90 |
| (mcU/mL) | TRF_{HMB} | 10 ± 2 | 13 ± 3 | 3 | 30 | 0.27 | | | |
| | TRF | 10 ± 2 | 12 ± 3 | 2 | 20 | 0.22 | | | |
| LDL | CD | 92 ± 7 | 99 ± 8 | 7 | 8 | 0.25 | 0.87 | 0.31 | 0.09 |
| (mg/dL) | TRF_{HMB} | 85 ± 7 | 97 ± 7 | 12 | 14 | 0.48 | | | |
| | TRF | 97 ± 7 | 89 ± 7 | −8 | −8 | −0.32 | | | |

Mean ± SE; P values from mixed model analysis
*Statistically significant difference between baseline and W 8 value in all groups combined
CD; control diet; ES: effect size; HDL: high-density lipoprotein cholesterol; I: interaction; ITT: intention-to-treat; LDL: low-density lipoprotein cholesterol; PP: per protocol; TRF: time-restricted feeding; TRF_{HMB}: time-restricted feeding plus beta-hydroxy beta-methylbutyrate supplementation; VLDL: very low-density lipoprotein cholesterol.

Supplemental Table 10.
Vascular Assessments.

| | PP | | | | | | | | |
|---|---|---|---|---|---|---|---|---|---|
| | | | | | Δ | ES | P | P | P |
| | Group | Baseline | W 8 | Δ | (%) | (d) | (group) | (time) | (I) |
| Brachial Systolic | CD | 107 ± 2 | 109 ± 2 | 2 | 2 | 0.31 | 0.34 | 0.76 | 0.51 |
| Pressure (mmHg) | TRF_{HMB} | 111 ± 2 | 112 ± 2 | 1 | 1 | 0.13 | | | |
| | TRF | 112 ± 2 | 110 ± 2 | −2 | −2 | −0.26 | | | |
| Brachial Diastolic | CD | 65 ± 2 | 64 ± 2 | −1 | −2 | −0.16 | 0.64 | 0.38 | 0.64 |
| Pressure (mmHg) | TRF_{HMB} | 67 ± 2 | 64 ± 2 | −3 | −4 | −0.49 | | | |
| | TRF | 66 ± 2 | 66 ± 2 | 0 | 0 | 0.05 | | | |
| Aortic Systolic | CD | 93 ± 2 | 94 ± 2 | 1 | 1 | 0.18 | 0.52 | 0.78 | 0.72 |
| Pressure (mmHg) | TRF_{HMB} | 97 ± 2 | 95 ± 2 | −2 | −2 | −0.20 | | | |
| | TRF | 96 ± 2 | 95 ± 2 | −1 | −1 | −0.16 | | | |
| Aortic Diastolic | CD | 66 ± 2 | 65 ± 2 | −1 | −2 | −0.20 | 0.70 | 0.33 | 0.65 |
| Pressure (mmHg) | TRF_{HMB} | 68 ± 2 | 65 ± 2 | −3 | −4 | −0.53 | | | |
| | IRF | 67 ± 2 | 67 ± 2 | 0 | 0 | 0.02 | | | |
| Heart Rate (bpm) | CD | 67 ± 4 | 61 ± 4 | −6 | −9 | −0.46 | 0.90 | 0.18 | 0.46 |
| | TRF_{HMB} | 62 ± 5 | 62 ± 5 | 0 | 0 | 0.06 | | | |
| | TRF | 63 ± 5 | 60 ± 4 | −3 | −5 | −0.29 | | | |
| Pulse Wave | CD | 6.1 ± 0.2 | 6.0 ± 0.2 | −0.1 | −2 | −0.28 | 0.29 | 0.47 | 0.12 |
| Velocity | TRF_{HMB} | 5.5 ± 0.3 | 5.8 ± 0.2 | 0.3 | 5 | 0.46 | | | |
| | TRF | 6.0 ± 0.2 | 6.1 ± 0.2 | 0.1 | 2 | 0.13 | | | |
| | | ITT | | | | | | | |
| | PP | | | | Δ | ES | P | P | P |
| | Group | Baseline | W 8 | Δ | (%) | (d) | (group) | (time) | (I) |
| Brachial Systolic | CD | 108 ± 2 | 109 ± 2 | 1 | 1 | 0.20 | 0.44 | 0.58 | 0.43 |
| Pressure (mmHg) | TRF_{HMB} | 112 ± 2 | 110 ± 2 | −2 | −2 | −0.22 | | | |
| | TRF | 113 ± 2 | 111 ± 2 | −2 | −2 | −0.28 | | | |
| Brachial Diastolic | CD | 64 ± 1 | 64 ± 2 | 0 | 0 | −0.13 | 0.41 | 0.10 | 0.48 |
| Pressure (mmHg) | TRF_{HMB} | 67 ± 1 | 63 ± 2 | −4 | −6 | −0.63 | | | |
| | TRF | 67 ± 1 | 66 ± 2 | −1 | −3 | −0.14 | | | |
| Aortic Systolic | CD | 94 ± 1 | 94 ± 2 | 0 | 0 | 0.12 | 0.57 | 0.29 | 0.44 |
| Pressure (mmHg) | TRF_{HMB} | 96 ± 2 | 94 ± 2 | −2 | −2 | −0.46 | | | |
| | TRF | 97 ± 1 | 95 ± 2 | −2 | −2 | −0.25 | | | |
| Aortic Diastolic | CD | 66 ± 1 | 65 ± 2 | −1 | −2 | −0.16 | 0.44 | 0.09 | 0.51 |
| Pressure (mmHg) | TRF_{HMB} | 68 ± 2 | 64 ± 2 | −4 | −6 | −0.64 | | | |
| | IRF | 68 ± 1 | 67 ± 2 | −1 | −1 | −0.16 | | | |
| Heart Rate (bpm) | CD | 68 ± 3 | 62 ± 3 | −6 | −9 | −0.51 | 0.81 | 0.07 | 0.24 |
| | TRF_{HMB} | 62 ± 3 | 63 ± 3 | 1 | 2 | 0.07 | | | |
| | TRF | 64 ± 3 | 60 ± 3 | −4 | −6 | −0.34 | | | |
| Pulse Wave | CD | 6.3 ± 0.2 | 6.1 ± 0.1 | −0.2 | −3 | −0.34 | 0.03* | 0.92 | 0.07 |
| Velocity | TRF_{HMB} | 5.5 ± 0.2 | 5.7 ± 0.1 | 0.2 | 4 | 0.37 | | | |
| | TRF | 5.9 ± 0.2 | 5.9 ± 0.1 | 0.0 | 0 | 0.00 | | | |

Mean ± SE; P values from mixed model analysis
*Group main effect indicating difference between CD and TRF_{HMB}
CD: control diet; ES: effect size; I: interaction; ITT: intention-to-treat; PP: per protocol; TRF: time-restricted feeding; TRF_{HMB}: time-restricted feeding plus beta-hydroxy beta-methylbutyrate supplementation; W 8: week 8.

Supplemental Table 11.
Cortisol Awakening Response.

| | PP | | | | | | | | |
|---|---|---|---|---|---|---|---|---|---|
| | | | | | Δ | ES | P | P | P |
| | Group | Baseline^{a} | W 8 | Δ | (%) | (d) | (group) | (time) | (I) |
| Cortisol Awakening | CD | 22.2 ± 3.1 | 19.9 ± 4.2 | −2.3 | −10 | −0.33 | 0.07 | 0.48 | 0.56 |
| Response (AUC) | TRF_{HMB} | 14.0 ± 3.3 | 18.2 ± 4.7 | 4.2 | 30 | 0.64 | | | |
| | TRF | 24.4 ± 3.1 | 28.6 ± 4.4 | 4.2 | 17 | 0.64 | | | |
| Average Cortisol | CD | 0.47 ± 0.07 | 0.45 ± 0.08 | 0.0 | −4 | −0.27 | 0.14 | 0.54 | 0.79 |
| (μg/dL) | TRF_{HMB} | 0.34 ± 0.07 | 0.42 ± 0.1 | 0.1 | 24 | 0.93 | | | |
| | TRF | 0.55 ± 0.07 | 0.59 ± 0.09 | 0.0 | 7 | 0.50 | | | |
| | | ITT | | | | | | | |
| | PP | | | | Δ | ES | P | P | P |
| | Group | Baseline^{b} | W 8 | Δ | (%) | (d) | (group) | (time) | (I) |
| Cortisol Awakening | CD | 19.4 ± 3.2 | 18.8 ± 4.1 | −0.6 | −3 | −0.04 | 0.26 | 0.96 | 0.90 |
| Response (AUC) | TRF_{HMB} | 17.5 ± 3.2 | 16.9 ± 4.0 | −0.6 | −3 | −0.05 | | | |
| | TRF | 23.0 ± 3.2 | 24.7 ± 3.7 | 1.7 | 7 | 0.14 | | | |
| Average Cortisol | CD | 0.42 ± 0.07 | 0.43 ± 0.08 | 0.01 | 2 | 0.04 | 0.55 | 0.52 | 0.65 |
| (μg/dL) | TRF_{HMB} | 0.40 ± 0.07 | 0.39 ± 0.98 | −0.01 | −3 | −0.04 | | | |
| | TRF | 0.52 ± 0.07 | 0.52 ± 0.08 | 0.00 | 0 | 0.00 | | | |

Mean ± SE; P values from mixed model analysis
^{a}At baseline, there were no statistically significant differences between groups for AUC (p = 0.08) or average cortisol (p = 0.13);
^{b}At baseline, there were no differences between groups for AUC (p = 0.48) or average cortisol (p = 0.43).
AUC: area under the curve; CD: control diet; ES: effect size; I: interaction; ITT: intention-to-treat; PP: per protocol; TRF: time-restricted feeding; TRF_{HMB}: time-restricted feeding plus beta-hydroxy beta-methylbutyrate supplementation; W 8: week 8.

Supplemental Table 12.
Questionnaire Responses.

| | PP | | | | | | | | | |
|---|---|---|---|---|---|---|---|---|---|---|
| | | | | | | Δ | P | P | P | ITT |
| | Group | Baseline | W 4 | W 8 | Δ | (%) | (group) | (time) | (I) | Baseline |
| MFQ | CD | 1.7 ± 0.6 | 1.4 ± 0.5 | 1.1 ± 0.5 | −0.6 | −35 | 0.17 | 0.005*^{a} | 0.26 | 1.7 ± 0.6 |
| | TRF_{HMB} | 1.1 ± 0.7 | 0.6 ± 0.5 | 0.1 ± 0.5 | −1.0 | −91 | | | | 1.4 ± 0.6 |
| | TRF | 2.8 ± 0.6 | 1.5 ± 0.5 | 1.9 ± 0.5 | −0.9 | −32 | | | | 2.9 ± 0.6 |
| PSQI | CD | 3.0 ± 0.3 | 2.7 ± 0.3 | 3.0 ± 0.3 | 0.0 | 0 | 0.50 | 0.45 | 0.42 | 3.1 ± 0.3 |
| | TRF_{HMB} | 3.1 ± 0.3 | 2.7 ± 0.3 | 2.6 ± 0.3 | −0.5 | −16 | | | | 3.1 ± 0.3 |
| | TRF | 3.1 ± 0.3 | 3.1 ± 0.3 | 3.3 ± 0.3 | 0.2 | 6 | | | | 3.2 ± 0.3 |
| TFEQ-CR | CD | 16.9 ± 1.1 | 17.0 ± 1.1 | 16.9 ± 0.9 | 0.0 | 0 | 0.44 | 0.22 | 0.53 | 17.1 ± 1 |
| | TRF_{HMB} | 17.1 ± 1.2 | 18.3 ± 1.2 | 18.8 ± 1.1 | 1.7 | 10 | | | | 17.8 ± 1 |
| | TRF | 18.5 ± 1.2 | 18 ± 1.1 | 19.4 ± 1.0 | 0.9 | 5 | | | | 17.7 ± 1 |
| TFEQ-UE | CD | 15.6 ± 1.1 | 17.6 ± 1.8 | 16.6 ± 1.6 | 1.0 | 6 | 0.84 | 0.08 | 0.08 | 16.6 ± 1.1 |
| | TRF_{HMB} | 18.4 ± 1.3 | 18.7 ± 2.0 | 15.5 ± 1.8 | −2.9 | −16 | | | | 20.0 ± 1.2 |
| | TRF | 18.8 ± 1.2 | 17.1 ± 1.9 | 17.0 ± 1.7 | −1.8 | −10 | | | | 19.1 ± 3.2 |
| TFEQ-EE | CD | 4.3 ± 0.5 | 5.2 ± 0.6 | 4.8 ± 0.6 | 0.5 | 12 | 0.72 | 0.22 | 0.21 | 4.6 ± 0.5 |
| | TRF_{HMB} | 4.9 ± 0.6 | 4.4 ± 0.7 | 3.7 ± 0.7 | −1.2 | −24 | | | | 5.4 ± 0 5 |
| | TRF | 5.4 ± 0.6 | 4.8 ± 0.6 | 4.8 ± 0.6 | −0.6 | −11 | | | | 5.7 ± 0.5 |

| | | ITT | | | | | | |
|---|---|---|---|---|---|---|---|---|
| | PP | | | | Δ | P | P | P |
| | Group | W 4 | W 8 | Δ | (%) | (group) | (time) | (I) |
| MFQ | CD | 1.2 ± 0.4 | 1.0 ± 0.4 | −0.7 | −41 | 0.14 | 0.001*^{a} | 0.58 |
| | TRF_{HMB} | 0.7 ± 0.4 | 0.4 ± 0.4 | −1.0 | −71 | | | |
| | TRF | 1.6 ± 0.4 | 1.7 ± 0.4 | −1.2 | −41 | | | |
| PSQI | CD | 2.9 ± 0.3 | 3.1 ± 0.3 | 0.0 | 0 | 0.88 | 0.38 | 0.63 |
| | TRF_{HMB} | 3.0 ± 0.3 | 2.7 ± 0.2 | −0.4 | −13 | | | |
| | TRF | 3.0 ± 0.3 | 3.0 ± 0.2 | −0.2 | −6 | | | |
| TFEQ-CR | CD | 16.9 ± 3 | 16.9 ± 3.3 | −0.2 | −1 | 0.73 | 0.52 | 0.69 |
| | TRF_{HMB} | 18.0 ± 1 | 18.2 ± 1.1 | 0.4 | 2 | | | |
| | TRF | 16.7 ± 1 | 18.1 ± 1.3 | 0.4 | 2 | | | |
| TFEQ-UE | CD | 17.3 ± 1.4 | 16.8 ± 1.3 | 0.2 | 1 | 0.59 | 0.01*^{b} | 0.20 |
| | TRF_{HMB} | 18.1 ± 1.4 | 16.3 ± 1.3 | −3.7 | −19 | | | |
| | TRF | 18.8 ± 1.4 | 17.3 ± 3.2 | −1.8 | −9 | | | |
| TFEQ-EE | CD | 4.9 ± 0.5 | 4.7 ± 0.6 | 0.1 | 2 | 0.59 | 0.25 | 0.76 |
| | TRF_{HMB} | 4.9 ± 0.5 | 4.5 ± 0.6 | −0.9 | −17 | | | |
| | TRF | 5.4 ± 0.5 | 5.1 ± 0.6 | −0.6 | −11 | | | |

Mean ± SE; P values from mixed model analysis
*Statistically significant (p < 0.05);
^{a}Values at W 4 and W 8 differed from baseline;
^{b}Value at W 8 differed from baseline
CD: control diet; CR: Cognitive Restraint; EE: Emotional Eating I: interaction; ITT: intention-to-treat; MFQ: Mood and Feelings Questionnaire; PP: per protocol; PSQI: Pittsburgh Sleep Quality Index; TFEQ: Three-Factor Eating Questionnaire; TRF: time-restricted feeding; TRF_{HMB}: time-restricted feeding plus beta-hydroxy beta-methylbutyrate supplementation, UE. Uncontrolled Eating, W 4: week 4; W 8: week 8.

Supplemental Table 13
Menstrual Cycle Analysis.

| | | PP | | | ITT | | |
|---|---|---|---|---|---|---|---|
| | | CD | TRF_{HMB} | TRF | CD | TRF_{HMB} | TRF |
| | | (n = 9) | (n = 7) | (n = 8) | (n = 14) | (n = 13) | (n = 13) |
| | Regular Cycles (%) | 78 | 57 | 63 | 79 | 77 | 69 |
| Baseline | Follicular Phase (%) | 44 | 29 | 13 | 29 | 39 | 31 |
| | Luteal Phase (%) | 22 | 43 | 50 | 43 | 46 | 39 |
| | Unknown (%) | 33 | 29 | 38 | 29 | 15 | 31 |
| W 4 | Follicular Phase (%) | 33 | 29 | 25 | 31 | 33 | 42 |
| Assessments | Luteal Phase (%) | 33 | 29 | 38 | 31 | 42 | 25 |
| | Unknown (%) | 33 | 43 | 38 | 39 | 25 | 33 |
| W 8 | Follicular Phase (%) | 33 | 17 | 38 | 33 | 44 | 25 |
| Assessments | Luteal Phase (%) | 33 | 50 | 38 | 33 | 44 | 50 |
| | Unknown (%) | 33 | 33 | 25 | 33 | 11 | 25 |

CD: control diet; ITT: intention-to-treat; PP: per protocol; TRF: time-restricted feeding; TRF_{HMB}: time-restricted feeding plus beta-hydroxy beta-methylbutyrate supplementation; W 4: week 4; W 8: week 8.

1. A method for promoting fat loss in an individual undergoing intermittent fasting, comprising administering to said individual a composition comprising from about 0.5 g to about 30 g of β-hydroxy-β-methylbutyrate (HMB).

2. The method of claim 1, wherein said HMB is selected from the group consisting of its free acid form, its salt, its ester and its lactone.

3. The method of claim 1, wherein said HMB is a calcium salt.

4. The method of claim 1, wherein HMB is in the free acid form.

5. The method of claim 1, wherein the intermittent fasting is time restricted feeding.

6. The method of claim 1, wherein the intermittent fasting is alternate day fasting.

7. A method of accelerating fat loss comprising the steps of administering from about 0.5 g to about 30 g of β-hydroxy-β-methylbutyrate (HMB) to an individual undergoing intermittent fasting.

8. The method of claim 7, wherein said HMB is selected from the group consisting of its free acid form, its salt, its ester and its lactone.

9. The method of claim 7, wherein said HMB is a calcium salt.

10. The method of claim 7, wherein HMB is in the free acid form.

11. The method of claim 7, wherein the intermittent fasting is time restricted feeding.

12. The method of claim 7, wherein the intermittent fasting is alternate day fasting.

13. A method of improving muscular performance in an individual undergoing intermittent fasting, comprising the steps of consuming from about 0.5 g to about 30 g of β-hydroxy-β-methylbutyrate (HMB).

14. The method of claim 13, wherein said HMB is selected from the group consisting of its free acid form, its salt, its ester and its lactone.

15. The method of claim 13, wherein said HMB is a calcium salt.

16. The method of claim 13, wherein HMB is in the free acid form.

17. The method of claim 13, wherein the intermittent fasting is time restricted feeding.

18. The method of claim 13, wherein the intermittent fasting is alternate day fasting.

19. A method of increasing fat free mass in an individual comprising the steps of administering from about 0.5 g to about 30 g of β-hydroxy-β-methylbutyrate (HMB) to an individual undergoing intermittent fasting.
